(19) United States
(12) Patent Application Publication
(10) Pub. No.: US 2010/0261780 A1
(43) Pub. Date: Oct. 14, 2010

(54) Highly Acidic Chitosan-Nucleic Acid Polyplex Compositions

(75) Inventors: Jun GAO, Coquitlam (CA); Carlos Fleet, North Vancouver (CA); Eric Hsu, Vancouver (CA); Anthony Cheung, Vancouver (CA)

Correspondence Address: ARNOLD & PORTER LLP;ATTN: IP DOCKETING DEPT., 555 TWELFTH STREET, N.W., WASHINGTON, DC, 20004-1206, US

(73) Assignee: enGene, Inc., Vancouver (CA)

(21) Appl. No.: 12/751,572

(22) Filed: Mar. 31, 2010

Related U.S. Application Data
(60) Provisional application No. 61/165,442, filed on Mar. 31, 2009.

(51) Int. Cl. A61K 31/7052; C08B 37/08; C12N 15/85; A61P 29/00; A61P 11/00

(52) U.S. Cl. 514/44 R; 536/20; 435/455

(57) ABSTRACT

The invention provides highly acidic chitosan-nucleic acid polyplex compositions. The compositions may be used to transfect cells in vitro and in vivo, and are particularly useful for transfecting cells of mucosal epithelia.

### CROSS-REFERENCE TO RELATED APPLICATIONS

This application claims the benefit of U.S. Ser. No. 61/165,442, filed 31 Mar. 2009, which is expressly incorporated herein in its entirety by reference.

### FIELD

The invention relates to highly acidic chitosan-nucleic acid polyplex compositions, as well as methods of making and using the same.

### BACKGROUND

Chitosan is a non-toxic cationic copolymer of N-acetyl-D-glucosamine and D-glucosamine. Chitosan can form a complex with nucleic acid and has been used as a DNA delivery vehicle to transfect cells.

Many biological applications of chitosan have involved the use of large chitosan polymers. Large chitosan polymers, on the order of hundreds to thousands of kilodaltons, are soluble only in acidic solutions. Dilute acetic acid is frequently used as a solvent for such large chitosans.

Low molecular weight chitosans, on the order of a few tens of kilodaltons or less, were originally thought to be too small to effectively package and protect DNA, and to serve as DNA delivery vehicles. However, several groups have more recently established that low molecular weight chitosans can be used to effectively package and protect DNA, and to serve as DNA delivery vehicles. Low molecular weight chitosans have been viewed as desirable for use as DNA delivery vehicles because they exhibit higher solubility at physiological pH, and a low pH environment is understood to promote the degradation of nucleic acid.

While high concentrations of nucleic acid are desirable for many purposes, there is difficulty in producing concentrated, stable dispersions of homogenous chitosan-nucleic acid complexes. Increasing the concentrations of chitosan and nucleic acid in a mixing solution leads to aggregation, instability, particle size variation, and precipitation.

The use of concurrent flow mixing to produce particles comprising DNA and condensing agents (e.g., polycationic carbohydrates) has been described (U.S. Pat. No. 6,537,813). To produce such particles, DNA solution and condensing agent solution may be concurrently and separately introduced into a flow-through mixer that comprises a static or dynamic mixer which provides for mixing and particle formation. The art teaches that maintaining the proper molar ratio of DNA and condensing agent throughout the introduction and mixing processes is important, and that a significant deviation from charge neutrality can lead to either incomplete condensation or particle aggregation in the process.

### SUMMARY OF THE INVENTION

The present inventors have found that highly acidic chitosan-nucleic acid polyplex compositions, having a pH well below that typically used to solubilize chitosan, exhibit a higher in vivo transfection efficiency of mucosal epithelium than polyplex compositions closer to physiological pH. The present compositions have a pH below 4.5, yet exhibit both stability and maintenance of nucleic acid integrity, and suitability for mucosal epithelium delivery. Paradoxically, low molecular weight chitosan, which has been developed in part for its solubility at a less acidic pH than high molecular weight chitosan, is particularly well suited for use in the present invention.

The present inventors have also overcome polyplex aggregation and precipitation problems to produce concentrated highly acidic chitosan-nucleic acid polyplex compositions that are stable. Further, the inventors have been able to produce concentrated preparations that are isotonic, which is highly desirable for pharmaceutical and therapeutic applications.

Accordingly, in one aspect, the invention provides highly acidic chitosan-nucleic acid polyplex compositions, comprising chitosan-nucleic acid polyplexes.

In a preferred embodiment, the subject compositions have a pH below 4.5, more preferably below 4.2, more preferably below 4.0, more preferably below 3.8.

In a preferred embodiment, the chitosan-nucleic acid polyplexes of the subject compositions comprise a therapeutic nucleic acid. In one embodiment, the therapeutic nucleic acid is a therapeutic RNA. In another embodiment, the therapeutic nucleic acid is a therapeutic nucleic acid construct encoding a therapeutic protein.

In a preferred embodiment, the subject composition is isotonic.

In a preferred embodiment, the subject composition is stable.

In a preferred embodiment, the subject composition is homogeneous. In a preferred embodiment, the subject composition has an average polydispersity index (“PDI”) of less than 0.5, more preferably less than 0.4, more preferably less than 0.3, and most preferably less than 0.2.

In a preferred embodiment, the subject composition is free of precipitated polyplex.

In a preferred embodiment, the subject composition has a nucleic acid concentration greater than 0.5 mg/ml, and is free of precipitated polyplex. More preferably, the subject composition has a nucleic acid concentration of at least 0.6 mg/ml, more preferably at least 0.75 mg/ml, more preferably at least 1.0 mg/ml, more preferably at least 1.2 mg/ml, and most preferably at least 1.5 mg/ml, and is free of precipitated polyplex.

In a preferred embodiment, the subject composition additionally comprises an aggregation inhibitor. In a preferred embodiment, the aggregation inhibitor is a sugar, preferably sucrose.

In a preferred embodiment, the polyplexes of the subject composition comprise chitosan molecules having on average less than 3000, more preferably less than 2000, more preferably less than 1500, more preferably less than 1000, more preferably less than 500, more preferably less than 300, more preferably less than 150, more preferably less than 100, more preferably less than 50, and most preferably less than 30 glucosamine monomer units.

In a preferred embodiment, the polyplexes of the subject composition have an N:P ratio of at least 2:1, more preferably at least 5:1, more preferably at least 10:1, more preferably at least 15:1, and most preferably at least 20:1.

In a preferred embodiment, the polyplexes of the subject composition comprise chitosan that has an average molecular weight of less than 500 kDa, more preferably less than 300 kDa, more preferably less than 250 kDa, more preferably less than 150 kDa, more preferably less than 100 kDa, more preferably less than 50 kDa, more preferably less than 25 kDa, more preferably less than 16 kDa, more preferably less than 8 kDa, and most preferably less than 5 kDa.

In a preferred embodiment, the polyplexes of the subject composition have an average diameter of less than 750 nm, more preferably less than 500 nm, more preferably less than 250 nm, more preferably less than 200 nm, and most preferably less than 150 nm.

In a preferred embodiment, the subject composition consists essentially of chitosan-nucleic acid polyplexes and an aggregation inhibitor.

In another preferred embodiment, the subject composition consists essentially of chitosan-nucleic acid polyplexes.

In one aspect, the invention provides pharmaceutical compositions, comprising highly acidic chitosan-nucleic acid polyplex compositions of the invention.

In a preferred embodiment, the pharmaceutical composition is isotonic. In other embodiments, the pharmaceutical composition may be hypertonic or hypotonic.

In one aspect, the invention provides a method of transfecting cells of a mucosal epithelium, comprising contacting the cells of a mucosal epithelium with a highly acidic chitosan-nucleic acid polyplex composition of the invention.

In a preferred embodiment, the mucosal epithelium is present in a tissue selected from the group consisting of gastrointestinal tract tissue, respiratory tract tissue, lung tissue, sinus cavity tissue, oral cavity tissue, urinary tract tissue, bladder tissue, vaginal tissue, uterine tissue, cervical tissue, eye tissue, esophagus tissue, salivary gland tissue, nasolaryngeal tissue, kidney tissue, and larynx/pharynx tissue.

In one aspect, the invention provides a method for treating a disease involving inflammation of a mucosal epithelium, comprising administering to a patient having a disease involving inflammation of a mucosal epithelium a therapeutically effective amount of a pharmaceutical composition of the invention. The subject pharmaceutical composition is preferably administered locally to the mucosal epithelium.

In a preferred embodiment, the subject pharmaceutical composition comprises a therapeutic nucleic acid construct encoding an anti-inflammatory protein. In one embodiment, the anti-inflammatory protein is a TNFα inhibitor. In another embodiment, the anti-inflammatory protein is an IL-1 inhibitor. In another preferred embodiment, the anti-inflammatory protein is IL-10.

In a preferred embodiment, the disease involving inflammation of a mucosal epithelium is inflammatory bowel disease (IBD). In another preferred embodiment, the disease involving inflammation of a mucosal epithelium is interstitial cystitis. In another preferred embodiment, the disease involving inflammation of a mucosal epithelium is chronic obstructive pulmonary disease (COPD). In another preferred embodiment, the disease involving inflammation of a mucosal epithelium is asthma.

### BRIEF DESCRIPTION OF THE DRAWINGS

FIG. 1. Pig plasma SEAP detected in response to administration of c150 chitosan-nucleic acid particles containing gWIZ-SEAP plasmid DNA. Drug product formulation for pH 4 was C(24,98)-N20-c150-Ac25-Suc9-pH4.0. Drug product formulation for pH 4.8 was C(24,98)-N20-c150-Ac25-Suc9-pH4.8.

FIG. 2. Exemplary Process Block for 1 L In-line Mixing Batch and TFF Concentration>Diafiltration>Concentration.

FIG. 3. Small-Scale In-line Mixing Schematic. Syringes are polypropylene (PP) latex-free and can be scaled up to 60 mL each. Two precision syringe pumps drive the syringes. Tubing is 1/16″ Pt-cured silicone. Mixing junction shown is a Y. Mixing junction material of construction is PP.

FIG. 4. Mid-Scale In-line Mixing Schematic for 10 L. Displayed schematic is for a 10 L batch. All vessels are scaled accordingly for smaller or larger batch sizes. Pt-cured tubing diameter, 0.48 cm ( 3/16″). Pump flow rates are indicated for a 2:1 DNA:chitosan volume mixing ratio.

FIG. 5. TFF Concentration & Diafiltration Schematic. TFF diafiltration scheme is shown. During TFF concentration, the dialysis buffer line is disconnected from the retentate vessel and replaced with an atmospheric vent filter.

FIG. 6. Modeling pH Shift during TFF Concentration. Each point indicates the relative volume-fold reduction (=increasing DNA concentration) of the polyplex. For example, the point labeled 2× is approximately c1200.

FIG. 7. Stability of Polyplex after Second TFF Concentration Step. Undiluted post-TFF sample was incubated at 25° C. and monitored for particle size every 2 hours.

FIG. 8. In-Process pH Data. TFF fraction codes on the X-axis are as follows: C1: TFF concentration step #1; D: TFF diafiltration, indicated in # of wash volumes (WV); C2: TFF concentration step #2.

FIG. 9. Transfection of mouse bladder in vivo. Naïve C57BL/6 mice were delivered with chitosan-DNA polyplexes C(24,98)-c1000-pH4 carrying EF1a-SEAP or control vehicle. After 2 days, mice were sacrificed and tissues were harvested. Relative increases in SEAP mRNA in bladder tissue of the treated mice over naïve mice (non-transfected) are shown.

FIG. 10. Effect of EG-10 (hIL-10) highly acidic chitosan-nucleic acid polyplex composition on body weight of chronic IBD mice. Each dose of highly acidic chitosan-nucleic acid polyplex composition was administered 7 days apart. Body weight of these mice were monitored weekly throughout the experiment and significant improvement in weight gain associated with the EG-10 treated group following each weekly treatment were observed.

FIG. 11. Effect of EG-10 (hIL-10) highly acidic chitosan-nucleic acid polyplex composition on three pro-inflammatory cytokines. Five days after the last treatment, mice from both groups were sacrificed and their colons were removed and pro-inflammatory cytokine levels were measured. The EG-10 treated mice resulted in reduced levels of IL-6 IL-1β and TNF-α mRNA when compared to SEAP treated mice.

FIG. 12. Agarose gel electrophoresis for two batches (DP-0089 and DP-0090) of final polyplex product from mid-scale manufacturing after 360 days at −80° C. Location of polyplex and DNA (supercoiled and nicked) are indicated. Drug product formulations were C(24,98)-N10-c1000-Ac70-Suc9-pH4.0.

### DETAILED DESCRIPTION

By “chitosan-nucleic acid polyplex”, “chitosan-nucleic acid polyplex particles”, “chitosan-nucleic acid complex”, “polyplex”, or grammatical equivalents, is meant a complex comprising a plurality of chitosan molecules and a plurality of nucleic acid molecules. Chitosan monomers include derivatives, including chitosan with attached ligand. “Derivatives” will be understood to include the broad category of chitosan-based polymers comprising covalently modified N-acetyl-D-glucosamine and/or D-glucosamine units, as well as chitosan-based polymers incorporating other units, or attached to other moieties. Derivatives are frequently based on a modification of the hydroxyl group or the amine group of glucosamine. Examples of chitosan derivatives include, but are not limited to, trimethylated chitosan, PEGylated chitosan, thiolated chitosan, galactosylated chitosan, alkylated chitosan, PEI-incorporated chitosan, arginine modified chitosan, uronic acid modified chitosan, and the like. For further teaching on chitosan derivatives, see, for example, pp. 63-74 of “Non-viral Gene Therapy”, K. Taira, K. Kataoka, T. Niidome (editors), Springer-Verlag Tokyo, 2005, ISBN 4-431-25122-7; Zhu et al., Chinese Science Bulletin, December 2007, vol. 52 (23), pp. 3207-3215; WO 2008/082282; and Varma et al., Carbohydrate Polymers 55 (2004) 77-93, each of which is expressly incorporated herein in its entirety by reference.

Dispersed systems consist of particulate matter, known as the dispersed phase, distributed throughout a continuous medium. A “dispersion” of chitosan-nucleic acid polyplexes is a composition comprising hydrated chitosan-nucleic acid polyplexes, wherein polyplexes are distributed throughout the medium.

As used herein, “average weight” of chitosan polymers refers to the weight average molecular weight.

By “counter anion” is meant an anion capable of electrostatic interaction with a charged chitosan amine or other cation in its place. Preferred counter anions include acetate ion and chloride ion.

As used herein, a “pre-concentration” dispersion is one that has not undergone the concentrating process to form a concentrated dispersion, as described herein.

As used herein, “free” of polyplex precipitate means that the composition is essentially free from particles that can be observed on visual inspection.

Chitosan may be prepared as disclosed in U.S. Ser. No. 11/694,852 filed 30 Mar. 2007, which is expressly incorporated herein in its entirety by reference.

Highly Acidic Chitosan-Nucleic Acid Polyplex Compositions

In one aspect, the invention provides highly acidic chitosan-nucleic acid polyplex compositions, comprising chitosan-nucleic acid polyplexes. The nucleic acid component of the chitosan-nucleic acid polyplex is encapsulated in the chitosan-nucleic acid polyplex. In a preferred embodiment, the chitosan-nucleic acid polyplexes of the subject compositions are homogeneous and stable in the compositions.

A composition comprising a plurality of chitosan-nucleic acid polyplexes that are “homogeneous” refers to a composition having a narrow distribution of polyplex sizes. This narrow distribution of polyplex sizes can be measured, for example, by the “polydispersity index” (PDI) of the composition. A preferred PDI for the subject compositions is less than 0.5, more preferably less than 0.4, more preferably less than 0.3, and most preferably less than 0.2.

A composition comprising a plurality of chitosan-nucleic acid polyplexes that are “stable” refers to a composition in which polyplexes remain size stable, i.e., tend not to increase in size or aggregate over time. In a preferred embodiment, a composition of the invention comprises polyplexes that increase in average diameter by less than 100%, more preferably less than 50%, and most preferably less than 25%, at room temperature for at least 6 hours, more preferably at least 12 hours, more preferably at least 24 hours, and most preferably at least 48 hours.

The chitosan-nucleic acid polyplexes of the subject compositions are preferably stable under cooled conditions. In a preferred embodiment, a composition of the invention comprises polyplexes that increase in average diameter by less than 100%, more preferably less than 50%, and most preferably less than 25%, at 2-8 degrees Celsius for at least 6 hours, more preferably at least 12 hours, more preferably at least 24 hours, and most preferably at least 48 hours.

The chitosan-nucleic acid polyplexes of the subject compositions are preferably stable under freeze-thaw conditions. In a preferred embodiment, a composition of the invention comprises polyplexes that increase in average diameter by less than 100%, more preferably less than 50%, and most preferably less than 25% at room temperature for at least 6 hours, more preferably at least 12 hours, more preferably at least 24 hours, and most preferably at least 48 hours following thaw from frozen at −20 to −80 degrees Celsius.

Encapsulation of nucleic acid in a chitosan-nucleic acid polyplex of the invention can be shown, for example, by retardation of nucleic acid in gel electrophoresis.

In a preferred embodiment, the subject compositions have a pH below 4.5, more preferably below 4.2, more preferably below 4.0, more preferably below 3.8.

In one embodiment, the subject compositions have a pH in the range of 3.5-4.5. In one embodiment, the subject compositions have a pH in the range of 3.6-4.2. In one embodiment, the subject compositions have a pH in the range of 3.8-4.2.

In a preferred embodiment, the polyplexes of the subject compositions comprise chitosan molecules having on average less than 3000, more preferably less than 2000, more preferably less than 1500, more preferably less than 1000, more preferably less than 500, more preferably less than 300, more preferably less than 150, more preferably less than 100, more preferably less than 50, and most preferably less than 30 glucosamine monomer units.

In a preferred embodiment, the polyplexes of the subject compositions comprise chitosan that has an average molecular weight of less than 500 kDa, more preferably less than 300 kDa, more preferably less than 250 kDa, more preferably less than 150 kDa, more preferably less than 100 kDa, more preferably less than 50 kDa, more preferably less than 25 kDa, more preferably less than 16 kDa, more preferably less than 8 kDa, and most preferably less than 5 kDa.

In a preferred embodiment, the chitosan components of the subject compositions have an average molecular weight between 3 kDa and 250 kDa.

In one embodiment, the chitosan components of the subject compositions have an average molecular weight greater than or equal to 250 kDa.

In one embodiment, the chitosan components of the subject compositions have an average molecular weight less than or equal to 3 kDa.

In a preferred embodiment, the polyplexes of the subject compositions have an average diameter of less than 750 nm, more preferably less than 500 nm, more preferably less than 250 nm, more preferably less than 200 nm, and most preferably less than 150 nm.

In one embodiment, the polyplexes of the subject compositions have an average diameter of more than 100 nm.

In one embodiment, the chitosan-nucleic acid polyplexes of the subject compositions have an N:P ratio between 2:1 and 100:1, more preferably 5:1 and 90:1, more preferably 10:1 and 90:1, and most preferably 20:1 and 90:1.

In a preferred embodiment, the chitosan-nucleic acid polyplexes of the subject compositions have an average zeta potential between +20 mV and +60 mV.

In one embodiment, the chitosan-nucleic acid polyplexes of the subject compositions have an average zeta potential less than or equal to +20 mV.

In one embodiment, the chitosan-nucleic acid polyplexes of the subject compositions have an average zeta potential greater than or equal to +60 mV.

In a preferred embodiment, the chitosan molecules of the polyplex have a degree of deacetylation greater than 70%, more preferably greater than 75%, more preferably greater than 80%, more preferably greater than 85%, more preferably greater than 90%, more preferably greater than 95%, and most preferably at least 98%.

In one embodiment, the chitosan molecules of the polyplex have a degree of deacetylation less than or equal to 70%.

In a preferred embodiment, the subject composition consists essentially of chitosan-nucleic acid polyplexes and an aggregation inhibitor. In addition to the subject polyplexes and aggregation inhibitor, such a composition may include counter anion and other excipients, but excludes other substances which materially affect the activity of the subject composition.

In a preferred embodiment, the subject composition consists essentially of chitosan-nucleic acid polyplexes. In addition to the subject polyplexes, such a composition may include counter anion and other excipients, but excludes other substances which materially affect the activity of the subject composition.

In a preferred embodiment, the subject composition does not include parabens. This is particularly desirable where the composition has a nucleic acid concentration of greater than 0.5 mg/ml.

In a preferred embodiment, the subject composition has a counter anion concentration of between 10-200 mM, with 60-100 mM being highly preferred. In a preferred embodiment, the counter anion is acetate.

In a preferred embodiment, the subject composition has a nucleic acid concentration greater than 0.5 mg/ml, and is free of precipitated polyplex. More preferably, the composition has a nucleic acid concentration of at least 0.6 mg/ml, more preferably at least 0.75 mg/ml, more preferably at least 1.0 mg/ml, more preferably at least 1.2 mg/ml, and most preferably at least 1.5 mg/ml, and is free of precipitated polyplex. In a preferred embodiment, the compositions are hydrated. In a preferred embodiment, the composition is substantially free of uncomplexed nucleic acid.

In a preferred embodiment, the chitosan-nucleic acid polyplex composition additionally comprises an aggregation inhibitor. The aggregation inhibitor is an agent that partially or completely reduces polyplex aggregation and/or precipitation and provides for concentrating chitosan-nucleic acid polyplexes by concentrating means, preferably through the use of tangential flow filtration (“TFF”). A highly preferred aggregation inhibitor is sucrose, though other aggregation inhibitors, such as other sugars that are capable of reducing polyplex precipitation and which provide for concentrating chitosan-nucleic acid polyplexes may be used. Examples of other aggregation inhibitors include, but are not limited to, trehalose, glycerol, fructose, glucose, and other reducing and non-reducing sugars.

In a preferred embodiment, the aggregation inhibitor used is sucrose. The concentration of sucrose in the chitosan-nucleic acid polyplex dispersion is preferably between about 3% and 20% by weight. Most preferably the concentration of sucrose provides for an isotonic composition.

In a preferred embodiment, the highly acidic chitosan-nucleic acid polyplex composition is isotonic. Achieving isotonicity, while maintaining polyplex stability, is highly desirable in formulating pharmaceutical compositions, and these preferred compositions are well suited to pharmaceutical formulation and therapeutic applications.

In other embodiments, the composition may be hypertonic or hypotonic.

Nucleic Acids

The highly acidic chitosan-nucleic acid polyplex compositions comprise a nucleic acid component and a chitosan component. A nucleic acid of the present invention will generally contain phosphodiester bonds, although in some cases nucleic acid analogs are included that may have alternate backbones or other modifications or moieties incorporated for any of a variety of purposes, e.g., stability and protection. Other analog nucleic acids contemplated include those with non-ribose backbones. In addition, mixtures of naturally occurring nucleic acids, analogs, and both can be made. The nucleic acids may be single stranded or double stranded or contain portions of both double stranded and single stranded sequence. Nucleic acids include but are not limited to DNA, RNA and hybrids where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. Nucleic acids include DNA in any form, RNA in any form, including triplex, duplex or single-stranded, anti-sense, siRNA, ribozymes, deoxyribozymes, polynucleotides, oligonucleotides, chimeras, microRNA, and derivatives thereof.

In one embodiment, the nucleic acid component comprises a therapeutic nucleic acid. Therapeutic nucleic acids include therapeutic RNAs, which are RNA molecules capable of exerting a therapeutic effect in a mammalian cell. Therapeutic RNAs include antisense RNAs, siRNAs, short hairpin RNAs, microRNAs, and enzymatic RNAs. Therapeutic nucleic acids include nucleic acids that form triplex molecules, protein binding nucleic acids, ribozymes, deoxyribozymes, and small nucleotide molecules.

Therapeutic nucleic acids also include nucleic acids encoding therapeutic proteins.

In a preferred embodiment, the nucleic acid component comprises a therapeutic nucleic acid construct. The therapeutic nucleic acid construct is a nucleic acid construct capable of exerting a therapeutic effect. Therapeutic nucleic acid constructs preferably comprise nucleic acids encoding therapeutic proteins, but can alternatively produce transcripts that are therapeutic RNAs. A therapeutic nucleic acid may be used to effect genetic therapy by serving as a replacement or enhancement for a defective gene or to compensate for lack of a particular gene product, by encoding a therapeutic product. A therapeutic nucleic acid may also inhibit expression of an endogenous gene. A therapeutic nucleic acid may encode all or a portion of a translation product, and may function by recombining with DNA already present in a cell, thereby replacing a defective gene or portion thereof. A therapeutic nucleic acid may also encode a portion of a protein. A therapeutic protein may exert its effect by inhibiting a gene product. In a preferred embodiment, the therapeutic nucleic acid is selected from those disclosed in U.S. Ser. No. 11/694,852, which is expressly incorporated herein in its entirety by reference. See also WO2008020318, which is expressly incorporated herein in its entirety by reference.

Therapeutic proteins contemplated for use in the present invention include, but are not limited to, hormones, enzymes, cytokines, chemokines, antibodies, growth factors, differentiation factors, factors influencing blood clot formation, factors influencing blood glucose levels, factors influencing glucose metabolism, factors influencing lipid metabolism, factors influencing blood cholesterol levels, factors influencing blood LDL or HDL levels, factors influencing cell apoptosis, factors influencing food intake, factors influencing energy expenditure, factors influencing appetite, factors influencing nutrient absorption, factors influencing inflammation, and factors influencing bone formation. Particularly preferred are therapeutic nucleic acids encoding insulin, leptin, glucagon antagonist, GLP-1, GLP-2, Ghrelin, cholecystokinin, growth hormone, clotting factors, PYY, erythropoietin, inhibitors of inflammation, IL-10, IL-17 antagonists, TNFα antagonists, IL-1 antagonists, growth hormone releasing hormone, or parathyroid hormone.

Especially preferred therapeutic proteins contemplated in the present invention are anti-inflammatory proteins. Anti-inflammatory proteins contemplated for use in the present invention include, but are not limited to, anti-inflammatory cytokines, as well as protein antagonists of pro-inflammatory molecules, such as pro-inflammatory cytokines. Exemplary anti-inflammatory proteins include IL-10 (e.g., Fedorak et al., 2000, Gastroenterology. 2000 December; 119(6):1473-82.; Whalen et al., 1999, J Immunol. 1999 Mar. 15; 162(6):3625-32); IL-1Ra (e.g., Arend et al., 1998, Annu Rev Immunol. 1998; 16:27-55; Makarov et al., 1996, Proc Natl Acad Sci USA. 1996 Jan. 9; 93(1):402-6); IL-1Ra-Ig (e.g., Ghivizzani et al., 1998, Proc Natl Acad Sci USA. 1998 Apr. 14; 95(8):4613-8); IL-4 (e.g., Hogaboam et al., 1997, J Clin Invest. 1997 Dec. 1; 100(11):2766-76); IL-17 soluble receptor (e.g., Zhang et al., 2006, Inflamm Bowel Dis. 2006 May; 12(5):382-8; Ye et al., 2001, The Journal of Experimental Medicine, Volume 194, Number 4, Aug. 20, 2001 519-528); IL-6 (e.g., Xing et al., 1998, J Clin Invest. 1998 Jan. 15; 101(2):311-20); IL-11 (e.g., Trepicchio et al., 1997, J Immunol. 1997 Dec. 1; 159(11):5661-70); IL-13 (e.g., Mulligan et al., 1997, J Immunol. 1997 Oct. 1; 159(7):3483-9; Muchamuel et al., 1997, J Immunol. 1997 Mar. 15; 158(6):2898-903); IL-18 soluble receptor (e.g., Aizawa et al., 1999, FEBS Lett. 1999 Feb. 26; 445(2-3):338-42); TNF-α soluble receptor (e.g., Watts et al., 1999, J Leukoc Biol. 1999 December; 66(6):1005-13); TNF-α receptor Ig (e.g., Ghivizzani et al., 1998, Proc Natl Acad Sci USA. 1998 Apr. 14; 95(8):4613-8); TGF-β (e.g., Song et al., 1998, J Clin Invest. 1998 Jun. 15; 101(12):2615-21; Giladi et al., 1994); IL-12 (e.g., Hogan et al., 1998, Eur J Immunol. 1998 February; 28(2):413-23); IFN-γ (e.g., Dow et al., 1999, Hum Gene Ther. 1999 Aug. 10; 10(12):1905-14); IL-4 soluble receptor (e.g., Steinke et al., 2001, Respir Res. 2001; 2(2):66-70. Epub 2001 Feb. 19).

Especially preferred anti-inflammatory proteins for use in the present invention include IL-10, protein antagonists of TNFα, and protein antagonists of IL-1.

Expression Control Regions

In a preferred embodiment, a polyplex of the invention comprises a therapeutic nucleic acid, which is a therapeutic construct, comprising an expression control region operably linked to a coding region. The therapeutic construct produces therapeutic nucleic acid, which may be therapeutic on its own, or may encode a therapeutic protein.

In some embodiments, the expression control region of a therapeutic construct possesses constitutive activity. In a number of preferred embodiments, the expression control region of a therapeutic construct does not have constitutive activity. This provides for the dynamic expression of a therapeutic nucleic acid. By “dynamic” expression is meant expression that changes over time. Dynamic expression may include several such periods of low or absent expression separated by periods of detectable expression. In a number of preferred embodiments, the therapeutic nucleic acid is operably linked to a regulatable promoter. This provides for the regulatable expression of therapeutic nucleic acids.

Expression control regions comprise regulatory polynucleotides (sometimes referred to herein as elements), such as promoters and enhancers, that influence expression of an operably linked therapeutic nucleic acid.

Expression control elements included herein can be from bacteria, yeast, plant, or animal (mammalian or non-mammalian). Expression control regions include full-length promoter sequences, such as native promoter and enhancer elements, as well as subsequences or polynucleotide variants which retain all or part of full-length or non-variant function (e.g., retain some amount of nutrient regulation or cell/tissue-specific expression). As used herein, the term “functional” and grammatical variants thereof, when used in reference to a nucleic acid sequence, subsequence or fragment, means that the sequence has one or more functions of native nucleic acid sequence (e.g., non-variant or unmodified sequence). As used herein, the term “variant” means a sequence substitution, deletion, or addition, or other modification (e.g., chemical derivatives such as modified forms resistant to nucleases).

As used herein, the term “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Some expression control regions confer regulatable expression to an operably linked therapeutic nucleic acid. A signal (sometimes referred to as a stimulus) can increase or decrease expression of a therapeutic nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

Numerous regulatable promoters are known in the art. Preferred inducible expression control regions include those comprising an inducible promoter that is stimulated with a small molecule chemical compound. In one embodiment, an expression control region is responsive to a chemical that is orally deliverable but not normally found in food. Particular examples can be found, for example, in U.S. Pat. Nos. 5,989,910; 5,935,934; 6,015,709; and 6,004,941.

In one embodiment, the therapeutic construct further comprises an integration sequence. In one embodiment, the therapeutic construct comprises a single integration sequence. In another embodiment, the therapeutic construct comprises a first and a second integration sequence for integrating the therapeutic nucleic acid or a portion thereof into the genome of a target cell. In a preferred embodiment, the integration sequence(s) is functional in combination with a means for integration that is selected from the group consisting of mariner, sleeping beauty, FLP, Cre, φC31, R, lambda, and means for integration from integrating viruses such as AAV, retroviruses, and lentiviruses.

In one embodiment, the subject composition further comprises a non-therapeutic construct in addition to a therapeutic construct, wherein the non-therapeutic construct comprises a nucleic acid sequence encoding a means for integration operably linked to a second expression control region. This second expression control region and the expression control region operably linked to the therapeutic nucleic acid may be the same or different. The encoded means for integration is preferably selected from the group consisting of mariner, sleeping beauty, FLP, Cre, φC31, R, lambda, and means for integration from integrating viruses such as AAV, retroviruses, and lentiviruses.

For further teaching, see WO2008020318, which is expressly incorporated herein in its entirety by reference.

Methods for Preparing Highly Acidic Chitosan-Nucleic Acid Polyplex Compositions

A composition of highly acidic chitosan-nucleic acid polyplexes is preferably prepared by inline mixing, though other methods, such as forming a mixing solution by dripping nucleic acid or chitosan solution into the other may be used. However, inline mixing provides for the preparation of a large volume of homogeneous chitosan-nucleic acid polyplexes, preferably having an average PDI less than 0.5, more preferably less than 0.4, more preferably less than 0.3, and most preferably less than 0.2. In a preferred embodiment, the dispersion has a pH between 3.5-5.5.

In-line mixing is a well-known process whereby two (or more) fluid streams are brought together into a single stream. Additional description of in-line mixing and the concentrating of chitosan-nucleic acid polyplexes is found in PCT/CA2008/001714, filed 26 Sep. 2008, and published as WO 2009/039657, which is expressly incorporated herein in its entirety by reference. For additional disclosure on inline mixing see, for example, U.S. Pat. Nos. 6,251,599 and 6,537,813, each of which is expressly incorporated herein in its entirety by reference.

The compositions may be complexed at the desired low pH, or may be complexed at a higher pH and pH-adjusted following complexation to form the desired highly acidic dispersion.

While mixers such as static mixers and dynamic mixers may be used, such devices lead to an increased PDI of complexes formed by the present methods. Accordingly, in preferred embodiments of the present invention, inline mixing is done without the use of such mixers.

In a preferred embodiment, a highly acidic high concentration chitosan-nucleic acid polyplex composition of the invention is produced by concentrating a pre-concentration dispersion of chitosan-nucleic acid polyplexes. In one embodiment, the pre-concentration dispersion has a pH below 4.8, preferably pH between 3.5-4.5. In another embodiment, the pre-concentration dispersion has a pH greater than 4.5. Concentrated product may be pH adjusted to a pH below 4.5. A pre-concentration dispersion preferably has a concentration less than 0.5 mg/ml.

In the present invention, tangential flow filtration (“TFF”) is the preferred means for concentrating a pre-concentration dispersion of chitosan-nucleic acid polyplexes. In TFF operation, a chitosan-nucleic acid polyplex dispersion is pumped across the surface of a semi-permeable membrane while pressure is applied toward the membrane to force a portion of the fluid through the membrane. Molecules that are smaller than the membrane pores are transported through the membrane pores and collected as permeate. Permeating solutes include but are not limited to salts, ions, sugars and microbial preservatives. Molecular entities that are too large to pass through the membrane pores, including the chitosan-nucleic acid polyplex, are retained in the stream and re-circulated as retentate. In TFF concentration operation, the permeate is removed while the retentate is open to the atmospheric pressure, resulting in a volume reduction of the retentate. Using TFF, polyplex concentration may be increased many fold, the result being a highly concentrated polyplex dispersion. In a preferred embodiment, the concentrated polyplex dispersion is isotonic.

In a preferred embodiment, the concentration process further comprises one or more diafiltration operations. Diafiltration is particularly preferred when using pre-concentration chitosan-nucleic acid polyplex compositions having a pH below 4.8, though it may be used with compositions having a pH higher than 4.8.

In TFF diafiltration operation, the permeate is constantly replenished by adding new buffer to the retentate, resulting in an exchange of buffer in the retentate. Using TFF diafiltration, polyplex may be buffer exchanged while maintaining polyplex concentration, the result being a polyplex dispersion with a new buffer.

In one embodiment, the TFF diafiltration operation is carried out on the pre-concentration dispersion of chitosan-nucleic acid polyplexes prior to TFF concentration to a concentrated polyplex dispersion. In a preferred embodiment, the TFF diafiltration operation is carried out on the concentrated dispersion of chitosan-nucleic acid polyplexes after TFF concentration to a concentrated polyplex dispersion. In a highly preferred embodiment, the TFF diafiltration operation is carried out during the TFF concentration operation. In this operation, the pre-concentration dispersion of chitosan-nucleic acid polyplexes is partially concentrated by TFF concentration, then subjected to TFF diafiltration, then further concentrated by TFF concentration. This results in a concentrated polyplex dispersion with a new buffer, which further promotes the stability of chitosan-nucleic acid polyplexes.

In one embodiment, the number of wash volumes for TFF diafiltration is preferably less than 40. In a preferred embodiment, the number of wash volumes for TFF diafiltration is preferably less than 20. In a more preferred embodiment, the number of wash volumes for TFF diafiltration is preferably less than 10. In a highly preferred embodiment, the number of wash volumes for TFF diafiltration is preferably less than 6.

In one embodiment, the number of TFF diafiltration operations to be carried out during the concentration operation is less than 5 and greater than 1. In a preferred embodiment, the number of TFF diafiltration operations to be carried out is 1.

In a preferred embodiment, the TFF diafiltration buffer comprises chitosan.

In a preferred embodiment, the TFF diafiltration buffer comprises chitosan and a counter anion, preferably acetate.

In a preferred embodiment, the TFF diafiltration buffer comprises chitosan, a counter anion, preferably acetate, and an aggregation inhibitor, preferably sucrose.

In a preferred embodiment, the pH of the concentrated chitosan-nucleic acid polyplex dispersion is adjusted to a lower pH by addition of a pH adjustment buffer.

In a preferred embodiment, the pH adjustment buffer comprises chitosan.

In a preferred embodiment, the pH adjustment buffer comprises chitosan and a counter anion, preferably acetate.

In a preferred embodiment, the pH adjustment buffer slightly dilutes the concentrated chitosan-nucleic acid polyplex, preferably less than 5%.

In a preferred embodiment, the pH adjustment buffer is added to the concentrated chitosan-nucleic acid polyplex within one hour of completion of the TFF concentration operation.

In a preferred embodiment, a pre-concentration chitosan-nucleic acid polyplex dispersion comprises a sugar, preferably sucrose. As described below, it was found that sucrose is an aggregation inhibitor that prevents aggregation of particles during the concentration process.

Methods of Use

In one aspect, the invention provides methods for transfecting cells of mucosal epithelium. The methods comprise contacting the cells of a mucosal epithelium with a highly acidic chitosan-nucleic acid polyplex composition of the invention. In one embodiment, the transfection is done in vitro. In another embodiment, the transfection is done in vivo. The subject compositions are suitable for administration to mucosal epithelia and exhibit a high transfection efficiency of mucosal epithelium cells, notwithstanding the highly acidic nature of the compositions.

In a preferred embodiment, the mucosal epithelium is present in a tissue selected from the group consisting of gastrointestinal tract tissue, respiratory tract tissue, lung tissue, sinus cavity tissue, oral cavity tissue, urinary tract tissue, bladder tissue, vaginal tissue, uterine tissue, cervical tissue, eye tissue, esophagus tissue, salivary gland tissue, nasolaryngeal tissue, kidney tissue, and larynx/pharynx tissue.

In one aspect, the invention provides methods for treating diseases involving inflammation of mucosal epithelium. The methods comprise administering to a patient having a disease involving inflammation of a mucosal epithelium a therapeutically effective amount of a pharmaceutical composition of the invention. The subject pharmaceutical composition is preferably administered locally to the mucosal epithelium. The subject pharmaceutical composition comprises a therapeutic nucleic acid that has anti-inflammatory activity.

In a preferred embodiment, the subject pharmaceutical composition comprises a therapeutic nucleic acid construct encoding an anti-inflammatory protein. In one embodiment, the anti-inflammatory protein is a TNFα inhibitor. In another embodiment, the anti-inflammatory protein is an IL-1 inhibitor. In another preferred embodiment, the anti-inflammatory protein is IL-10.

In one embodiment, the therapeutic nucleic acid is a therapeutic RNA directed at a pro-inflammatory cytokine. Especially preferred are siRNAs directed at pro-inflammatory cytokines.

In a preferred embodiment, the disease involving inflammation of a mucosal epithelium is IBD. In another preferred embodiment, the disease involving inflammation of a mucosal epithelium is interstitial cystitis. In another preferred embodiment, the disease involving inflammation of a mucosal epithelium is chronic obstructive pulmonary disease (COPD). In another preferred embodiment, the disease involving inflammation of a mucosal epithelium is asthma.

The subject compositions are well suited for use in treating diseases or conditions that are treatable by transfection of mucosal epithelial cells. Such diseases include but are not limited to diseases that involve mucosal epithelial tissue. The subject compositions can also be used to treat diseases and conditions that do not involve the mucosal epithelial tissue to which the compositions may be administered. Such conditions and diseases are nonetheless therapeutically accessible through such transfection of mucosal epithelial tissue. For example, see WO2008020318, which is expressly incorporated herein in its entirety by reference. For example, administration of the subject compositions to the mucosal epithelium of the gut may be used to deliver an encoded therapeutic protein systemically.

A therapeutic nucleic acid may be used to effect genetic therapy by serving as a replacement or enhancement for a defective gene or to compensate for lack of a particular gene product, by encoding a therapeutic product. A therapeutic nucleic acid may also inhibit expression of an endogenous gene. A therapeutic nucleic acid may encode all or a portion of a translation product, and may function by recombining with DNA already present in a cell, thereby replacing a defective gene or portion thereof. A therapeutic nucleic acid may also encode a portion of a protein. A therapeutic protein may exert its effect by inhibiting a gene product.

Diseases or conditions that may be treated include, but are not limited to, diabetes, obesity, hormone deficiency, inflammatory bowel disease, diarrhea, irritable bowel syndrome, GI infection, peptic ulcers, gastroesophageal reflux, gastriparesis, hemorrhoids, malabsorption of nutrients, pancreatitis, hemochromatosis, celiac disease, macular degeneration, age-related macular degeneration, uveitis, retinitis pigmentosa, iritis, scleritis, glaucoma, keratititis, retinopathy, eye infection (e.g. keratomycosis), infections, endometriosis, cervicitis, urologic pain, polyps, fibroids, endometrial hyperplasia, urinary incontinence, bladder and urinary tract infection, overactive bladder, erectile dysfunction, diabetic neuropathy, diabetic nephropathy, membranous nephropathy, hypertension, food allergy, asthma, polycystic kidney disease, glomerulonephritis, dyslipidemia/hypercholesterolemia, metabolic syndrome, psoriasis, acne, rosacea, granulomatous dermatitis, wrinkles, depigmentation, chronic obstructive pulmonary disease, respiratory tract infection, cystic fibrosis, pulmonary vascular diseases, fibrosis, Huntington's disease, Alzheimer disease, Parkinson's disease, neurological disorders, autoimmune disease, metabolic syndromes, atherosclerosis, and inflammation. The methods comprise administering a therapeutically effective amount of a pharmaceutical composition of the invention to a patient.

Therapeutic proteins of the invention may be produced by the subject compositions comprising therapeutic nucleic acids encoding such therapeutic proteins. The use of therapeutic proteins described below refers to use of the subject compositions to effect such therapeutic protein use.

Therapeutic proteins contemplated for use in the invention have a wide variety of activities and find use in the treatment of a wide variety of disorders. The following description of therapeutic protein activities, and indications treatable with therapeutic proteins of the invention, is exemplary and not intended to be exhaustive. The term “subject” refers to an animal, with mammals being preferred, and humans being especially preferred. In embodiments wherein the therapeutic protein is an antagonist of a target protein, alternative therapeutic embodiments may employ therapeutic RNAs targeting the same target protein.

A partial list of therapeutic proteins and target diseases is shown in the following Table.

| | | | THERAPEUTIC |
|---|---|---|---|
| PROTEINS | TARGET DISEASE | FUNCTION | EFFECT |
| Insulin | Diabetes | Insulin replacement | Improve glucose |
| | | | tolerance. |
| | | | Delay/prevent |
| | | | diabetes. |
| Glucagon antagonists | Diabetes | Reduce endogenous | Improve glucose |
| | | glucose production | tolerance |
| GLP-1 | Diabetes | Stimulate growth of β- | Improve glucose |
| | Obesity | cells, improve insulin | tolerance. |
| | | sensitivity, suppress | Induce weight loss |
| | | appetite | |
| Leptin | Obesity | Appetite suppression | Induce weight loss. |
| | Diabetes | and improvement of | Improve glucose |
| | | insulin sensitivity | tolerance |
| CCK | Obesity | Appetite suppression | Induce weight loss |
| Growth Hormone | GH deficiencies, | GH replacement | Improve growth |
| (GH) | wasting and anti-aging | | |
| Clotting factors | Hemophilia | Clotting factors | Improve clotting time |
| | | replacement | |
| Therapeutic | Infections | Pathogen | Prevent infections or |
| antibodies and | | neutralization or | transplant rejections |
| antibody | | immune modulations | |
| fragments/portions | | | |
| Inflammation | Gastrointestinal organ | Immune modulation, | Prevent inflammation |
| inhibitors, e.g., IL-10, | inflammation; e.g., | modulation of | in target tissue |
| TNFα antagonists, IL- | inflammatory bowel | inflammation | |
| 17 antagonists, IL-1 | disease; bladder | | |
| antagonists | inflammation, e.g., | | |
| | interstitial cystitis; lung | | |
| | inflammation, e.g., | | |
| | chronic obstructive | | |
| | pulmonary disease | | |
| | (COPD); asthma | | |
| Pathogenic antigens | Infections | Vaccination against | Prevent or minimize |
| (e.g. Rotavirus, HIV, | Autoimmune diseases | pathogens and | infection by |
| SARS, anthrax, | | induction of immune | pathogen. |
| influenza) | | tolerance towards self- | Prevent allergic |
| Self-antigens (e.g. | | antigens or allergens | reactions or immune- |
| GAD, insulin, myelin, | | | reaction against self- |
| collagen) | | | antigens |
| Allergens (e.g. Arah-1 | | | |
| to 8, | | | |

Inflammatory Disorders

In a preferred embodiment, a therapeutic polypeptide of the present invention is used to modulate inflammation. For example, the therapeutic polypeptide may inhibit the proliferation and differentiation of cells involved in an inflammatory response. These molecules can be used to treat inflammatory conditions, both chronic and acute conditions, including inflammation associated with infection (e.g. septic shock, sepsis, or systemic inflammatory response syndrome (SIRS)), ischemia-reperfusion injury, endotoxin lethality, arthritis, pancreatitis, complement-mediated hyperacute rejection, nephritis, cytokine or chemokine induced lung injury, inflammatory bowel disease, chronic obstructive pulmonary disease (COPD), interstitial cystitis, Crohn's disease, or other diseases resulting from over production of pro-inflammatory cytokines (e.g. TNFα and IL-1).

In an especially preferred embodiment, the invention provides methods for treating diseases involving inflammation of mucosal epithelium. The methods comprise administering to a patient having a disease involving inflammation of a mucosal epithelium a therapeutically effective amount of a pharmaceutical composition of the invention. The subject pharmaceutical composition is preferably administered locally to the mucosal epithelium. In one embodiment, the subject pharmaceutical composition comprises a therapeutic nucleic acid construct encoding an anti-inflammatory protein. Anti-inflammatory proteins contemplated for use in the present invention include, but are not limited to, anti-inflammatory cytokines, as well as protein antagonists of pro-inflammatory molecules, such as pro-inflammatory cytokines. Exemplary anti-inflammatory proteins include IL-10 (e.g., Fedorak et al., 2000, Gastroenterology. 2000 December; 119(6):1473-82.; Whalen et al., 1999, J Immunol. 1999 Mar. 15; 162(6):3625-32); IL-1Ra (e.g., Arend et al., 1998, Annu Rev Immunol. 1998; 16:27-55; Makarov et al., 1996, Proc Natl Acad Sci USA. 1996 Jan. 9; 93(1):402-6); IL-1Ra-Ig (e.g., Ghivizzani et al., 1998, Proc Natl Acad Sci USA. 1998 Apr. 14; 95(8):4613-8); IL-4 (e.g., Hogaboam et al., 1997, J Clin Invest. 1997 Dec. 1; 100(11):2766-76); IL-17 soluble receptor (e.g., Zhang et al., 2006, Inflamm Bowel Dis. 2006 May; 12(5):382-8; Ye et al., 2001, The Journal of Experimental Medicine, Volume 194, Number 4, Aug. 20, 2001 519-528); IL-6 (e.g., Xing et al., 1998, J Clin Invest. 1998 Jan. 15; 101(2):311-20); IL-11 (e.g., Trepicchio et al., 1997, J Immunol. 1997 Dec. 1; 159(11):5661-70); IL-13 (e.g., Mulligan et al., 1997, J Immunol. 1997 Oct. 1; 159(7):3483-9; Muchamuel et al., 1997, J Immunol. 1997 Mar. 15; 158(6):2898-903); IL-18 soluble receptor (e.g., Aizawa et al., 1999, FEBS Lett. 1999 Feb. 26; 445(2-3):338-42); TNF-α soluble receptor (e.g., Watts et al., 1999, J Leukoc Biol. 1999 December; 66(6):1005-13); TNF-α receptor Ig (e.g., Ghivizzani et al., 1998, Proc Natl Acad Sci USA. 1998 Apr. 14; 95(8):4613-8); TGF-β (e.g., Song et al., 1998, J Clin Invest. 1998 Jun. 15; 101(12):2615-21; Giladi et al., 1994); IL-12 (e.g., Hogan et al., 1998, Eur J Immunol. 1998 February; 28(2):413-23); IFN-γ (e.g., Dow et al., 1999, Hum Gene Ther. 1999 Aug. 10; 10(12):1905-14); IL-4 soluble receptor (e.g., Steinke et al., 2001, Respir Res. 2001; 2(2):66-70. Epub 2001 Feb. 19).

In a preferred embodiment, the anti-inflammatory protein is a TNFα inhibitor. In another preferred embodiment, the anti-inflammatory protein is an IL-1 inhibitor. In another preferred embodiment, the anti-inflammatory protein is IL-10.

In a preferred embodiment, the disease involving inflammation of a mucosal epithelium is IBD. In another preferred embodiment, the disease involving inflammation of a mucosal epithelium is interstitial cystitis. In another preferred embodiment, the disease involving inflammation of a mucosal epithelium is chronic obstructive pulmonary disease (COPD). In another preferred embodiment, the disease involving inflammation of a mucosal epithelium is asthma.

Hyperglycemia and Body Mass

Therapeutic proteins include insulin and insulin analogs. Diabetes mellitus is a debilitating metabolic disease caused by absent (type 1) or insufficient (type 2) insulin production from pancreatic β-cells (Unger, R. H. et al, Williams Textbook of Endocrinology Saunders, Philadelphia (1998)). Beta-cells are specialized endocrine cells that manufacture and store insulin for release following a meal (Rhodes, et. al. J. Cell Biol. 105:145 (1987)) and insulin is a hormone that facilitates the transfer of glucose from the blood into tissues where it is needed. Patients with diabetes must frequently monitor blood glucose levels and many require multiple daily insulin injections to survive. However, such patients rarely attain ideal glucose levels by insulin injection (Turner, R. C. et al. JAMA 281:2005 (1999)). Furthermore, prolonged elevation of insulin levels can result in detrimental side effects such as hypoglycemic shock and desensitization of the body's response to insulin. Consequently, diabetic patients still develop long-term complications, such as cardiovascular diseases, kidney disease, blindness, nerve damage and wound healing disorders (UK Prospective Diabetes Study (UKPDS) Group, Lancet 352, 837 (1998)).

Disorders treatable by a method of the invention include a hyperglycemic condition, such as insulin-dependent (type 1) or -independent (type 2) diabetes, as well as physiological conditions or disorders associated with or that result from the hyperglycemic condition. Thus, hyperglycemic conditions treatable by a method of the invention also include a histopathological change associated with chronic or acute hyperglycemia (e.g., diabetes). Particular examples include degeneration of pancreas (β-cell destruction), kidney tubule calcification, eye damage (diabetic retinopathy), diabetic foot, ulcerations in mucosa such as mouth and gums, excess bleeding, delayed blood coagulation or wound healing and increased risk of coronary heart disease, stroke, peripheral vascular disease, dyslipidemia, hypertension and obesity.

The subject compositions are useful for decreasing glucose, improving glucose tolerance, treating a hyperglycemic condition (e.g., diabetes) or for treating a physiological disorders associated with or resulting from a hyperglycemic condition. Such disorders include, for example, diabetic neuropathy (autonomic), nephropathy (kidney damage), skin infections and other cutaneous disorders, slow or delayed healing of injuries or wounds (e.g., that lead to diabetic carbuncles), eye damage (retinopathy, cataracts) which can lead to blindness, diabetic foot and accelerated periodontitis. Such disorders also include increased risk of developing coronary heart disease, stroke, peripheral vascular disease, dyslipidemia, hypertension and obesity.

As used herein, the term “hyperglycemic” or “hyperglycemia,” when used in reference to a condition of a subject, means a transient or chronic abnormally high level of glucose present in the blood of a subject. The condition can be caused by a delay in glucose metabolization or absorption such that the subject exhibits glucose intolerance or a state of elevated glucose not typically found in normal subjects (e.g., in glucose-intolerant subdiabetic subjects at risk of developing diabetes, or in diabetic subjects). Fasting plasma glucose (FPG) levels for normoglycemia are less than about 110 mg/dl, for impaired glucose metabolism, between about 110 and 126 mg/dl, and for diabetics greater than about 126 mg/dl.

Disorders treatable by producing a protein in a gut mucosal tissue also include obesity or an undesirable body mass. Leptin, cholecystokinin, PYY and GLP-1 decrease hunger, increase energy expenditure, induce weight loss or provide normal glucose homeostasis. Thus, in various embodiments, a method of the invention for treating obesity or an undesirable body mass, or hyperglycemia, involves the use of a therapeutic nucleic acid encoding leptin, cholecystokinin, PYY or GLP-1. Disorders treatable also include those typically associated with obesity, for example, abnormally elevated serum/plasma LDL, VLDL, triglycerides, cholesterol, plaque formation leading to narrowing or blockage of blood vessels, increased risk of hypertension/stroke, coronary heart disease, etc. Ghrelin increases appetite and hunger. Thus, in various embodiments, a method of the invention for treating obesity or an undesirable body mass, or hyperglycemia, involves the use of an antagonist of ghrelin. In one embodiment, the antagonist is a therapeutic RNA targeting ghrelin.

As used herein, the term “obese” or “obesity” refers to a subject having at least a 30% increase in body mass in comparison to an age and gender matched normal subject. “Undesirable body mass” refers to subjects having 1%-29% greater body mass than a matched normal subject as well as subjects that are normal with respect to body mass but who wish to decrease or prevent an increase in their body mass.

In one embodiment, a therapeutic protein of the invention is a glucagon antagonist. Glucagon is a peptide hormone produced by α-cells in pancreatic islets and is a major regulator of glucose metabolism (Unger R. H. & Orci L. N. Eng. J. Med. 304:1518 (1981); Unger R. H. Diabetes 25:136 (1976)). As with insulin, blood glucose concentration mediates glucagon secretion. However, in contrast to insulin glucagon is secreted in response to a decrease in blood glucose. Therefore, circulating concentrations of glucagon are highest during periods of fast and lowest during a meal. Glucagon levels increase to curtail insulin from promoting glucose storage and stimulate liver to release glucose into the blood. A specific example of a glucagon antagonist is [des-His¹, des-Phe⁶, Glu⁹]glucagon-NH₂. In streptozotocin diabetic rats, blood glucose levels were lowered by 37% within 15 min of an intravenous bolus (0.75 μg/g body weight) of this glucagon antagonist (Van Tine B. A. et. al. Endocrinology 137:3316 (1996)). Additionally, in various embodiments, methods of the invention for treating diabetes, or hyperglycemia, involve the use of a therapeutic RNA to decrease the levels of glucagon production from the pancreas.

In another embodiment, a therapeutic protein of the invention useful for treating a hyperglycemic condition or undesirable body mass (e.g., obesity) is a glucagon-like peptide-1 (GLP-1). GLP-1 is a hormone released from L-cells in the intestine during a meal which stimulates pancreatic 3-cells to increase insulin secretion. GLP-1 has additional activities which make it an attractive therapeutic agent for treating obesity and diabetes. For example, GLP-1 reduces gastric emptying, suppresses appetite, reduces glucagon concentration, increases β-cell mass, stimulates insulin biosynthesis and secretion in a glucose-dependent fashion, and likely increases tissue sensitivity to insulin (Kieffer T. J., Habener J. F. Endocrin. Rev. 20:876 (2000)). Therefore, regulated release of GLP-1 in the gut to coincide with a meal can provide therapeutic benefit for a hyperglycemic condition or an undesirable body mass. GLP-1 analogs that are resistant to dipeptidyl peptidate IV (DPP IV) provide longer duration of action and improved therapeutic value. Thus, GLP-1 analogs are preferred therapeutic polypeptides. Additionally, in various embodiments, a method of the invention for treating diabetes, or hyperglycemia, involves the use of a DPP IV antagonist. In one embodiment, the antagonist is a therapeutic RNA targeting DPP IV.

In another embodiment, a therapeutic protein of the invention useful for treating a hyperglycemic condition is an antagonist to the hormone resistin. Resistin is an adipocyte-derived factor for which expression is elevated in diet-induced and genetic forms of obesity. Neutralization of circulating resistin improves blood glucose and insulin action in obese mice. Conversely, administration of resistin in normal mice impairs glucose tolerance and insulin action (Steppan C M et. al. Nature 409:307 (2001)). Production of a protein that antagonizes the biological effects of resistin in gut can therefore provide an effective therapy for obesity-linked insulin resistance and hyperglycemic conditions. Additionally, in various embodiments, methods of the invention for treating diabetes, or hyperglycemia, involve the use of a therapeutic RNA to decrease the levels of resistin expression in adipose tissue.

In another embodiment, a therapeutic polypeptide of the invention useful for treating a hyperglycemic condition or undesirable body mass (e.g., obesity) is leptin. Leptin, although produced primarily by fat cells, is also produced in smaller amounts in a meal-dependent fashion in the stomach. Leptin relays information about fat cell metabolism and body weight to the appetite centers in the brain where it signals reduced food intake (promotes satiety) and increases the body's energy expenditure.

In another embodiment, a therapeutic polypeptide of the invention useful for treating a hyperglycemic condition or undesirable body mass (e.g., obesity) is the C-terminal globular head domain of adipocyte complement-related protein (Acrp30). Acrp30 is a protein produced by differentiated adipocytes. Administration of a proteolytic cleavage product of Acrp30 consisting of the globular head domain to mice leads to significant weight loss (Fruebis J. et al. Proc. NatL Acad. Sci USA 98:2005 (2001)).

In another embodiment, a therapeutic polypeptide of the invention useful for treating a hyperglycemic condition or undesirable body mass (e.g., obesity) is cholecystokinin (CCK). CCK is a gastrointestinal peptide secreted from the intestine in response to particular nutrients in the gut. CCK release is proportional to the quantity of food consumed and is believed to signal the brain to terminate a meal (Schwartz M. W. et. al. Nature 404:661-71 (2000)). Consequently, elevated CCK can reduce meal size and promote weight loss or weight stabilization (i.e., prevent or inhibit increases in weight gain).

Regarding PYY, see for example 1e Roux et al., Proc Nutr Soc. 2005 May; 64(2):213-6.

Immunological Disorders

In one embodiment, a therapeutic protein of the invention possesses immunomodulatory activity. For example, a therapeutic polypeptide of the present invention may be useful in treating deficiencies or disorders of the immune system, by activating or inhibiting the proliferation, differentiation, or mobilization (chemotaxis) of immune cells. Immune cells develop through the process of hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, infectious, or other.

A therapeutic polypeptide of the present invention may be useful in treating deficiencies or disorders of hematopoietic cells. A therapeutic polypeptide of the present invention could be used to increase differentiation or proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

A therapeutic polypeptide of the present invention may also be useful in treating autoimmune disorders. Many autoimmune disorders result from inappropriate recognition of self as foreign material by immune cells. This inappropriate recognition results in an immune response leading to the destruction of the host tissue. Therefore, the administration of a therapeutic polypeptide of the present invention that inhibits an immune response, particularly the proliferation, differentiation, or chemotaxis of T-cells, may be an effective therapy in preventing autoimmune disorders.

Examples of autoimmune disorders that can be treated by the present invention include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin-dependent diabetes mellitis, Crohn's disease, ulcerative colitis, and autoimmune inflammatory eye disease.

Similarly, allergic reactions and conditions, such as asthma (particularly allergic asthma) or other respiratory problems, may also be treated by a therapeutic polypeptide of the present invention. Moreover, these molecules can be used to treat anaphylaxis, hypersensitivity to an antigenic molecule, or blood group incompatibility.

A therapeutic polypeptide of the present invention may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of a therapeutic polypeptide of the present invention that inhibits an immune response, particularly the proliferation, differentiation, or chemotaxis of T-cells, may be an effective therapy in preventing organ rejection or GVHD.

Clotting Disorders

In some embodiments, a therapeutic polypeptide of the present invention may also be used to modulate hemostatic (the stopping of bleeding) or thrombolytic activity (clot formation). For example, by increasing hemostatic or thrombolytic activity, a therapeutic polypeptide of the present invention could be used to treat blood coagulation disorders (e.g. afibrinogenemia, factor deficiencies), blood platelet disorders (e.g. thrombocytopenia), or wounds resulting from trauma, surgery, or other causes. Alternatively, a therapeutic polypeptide of the present invention that can decrease hemostatic or thrombolytic activity could be used to inhibit or dissolve clotting. These molecules could be important in the treatment of heart attacks (infarction), strokes, or scarring. In one embodiment, a therapeutic polypeptide of the invention is a clotting factor, useful for the treatment of hemophilia or other coagulation/clotting disorders (e.g., Factor VIII, IX or X)

Infectious Disease

In one embodiment, a therapeutic polypeptide of the present invention can be used to treat infectious disease. For example, by increasing the immune response, particularly increasing the proliferation and differentiation of B and/or T cells, infectious diseases may be treated. The immune response may be increased by either enhancing an existing immune response, or by initiating a new immune response. Alternatively, the therapeutic polypeptide of the present invention may also directly inhibit the infectious agent, without necessarily eliciting an immune response.

Viruses are one example of an infectious agent that can cause disease or symptoms that can be treated by a therapeutic polypeptide of the present invention. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Herpesviridae (such as, Cytomegalovirus, Herpes Simplex, Herpes Zoster), Mononegavirus (e.g. Paramyxoviridae, Morbillivirus, Rhabdoviridae), Orthomyxoviridae (e.g. Influenza), Papovaviridae, Parvoviridae, Picornaviridae, Poxyiridae (such as Smallpox or Vaccinia), Reoviridae (e.g. Rotavirus), Retroviridae (HTLV-I, HTLV-II, Lentivirus), and Togaviridae (e.g. Rubivirus). Viruses falling within these families can cause a variety of diseases or symptoms, including: arthritis, bronchiollitis, encephalitis, eye infections (e.g. conjunctivitis, keratitis), chronic fatigue syndrome, meningitis, opportunistic infections (e.g. AIDS), pneumonia, chickenpox, hemorrhagic fever, Measles, Mumps, Parainfluenza, Rabies, the common cold, Polio, Rubella, sexually transmitted diseases, skin diseases (e.g. Kaposi's, warts), and viremia. A therapeutic polypeptide of the present invention can be used to treat any of these symptoms or diseases.

Similarly, bacterial or fungal agents that can cause disease or symptoms and that can be treated or detected by a therapeutic polypeptide of the present invention include, but are not limited to, the following Gram-Negative and Gram-positive bacterial families and fungi: Actinomycetales (e.g. Corynebacterium, Mycobacterium, Norcardia), Aspergillosis, Bacillaceae (e.g. Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g. Acinetobacter, Gonorrhea, Menigococcal), Pasteurellacea Infections (e.g. Actinobacillus, Heamophilus, Pasteurella), Pseudomonas, Rickettsiaceae, Chlamydiaceae, Syphilis, and Staphylococcal. These bacterial or fungal families can cause the following diseases or symptoms: bacteremia, endocarditis, eye infections (conjunctivitis, tuberculosis, uveitis), gingivitis, opportunistic infections (e.g. AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g. cellulitis, dermatocycoses), toxemia, urinary tract infections, wound infections. A therapeutic polypeptide of the present invention can be used to treat any of these symptoms or diseases.

Moreover, parasitic agents causing disease or symptoms that can be treated by a therapeutic polypeptide of the present invention include, but are not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including: Scabies, Trombiculiasis, eye infections, intestinal disease (e.g. dysentery, giardiasis), lung disease, opportunistic infections (e.g. AIDS related), Malaria, pregnancy complications, and toxoplasmosis. A therapeutic polypeptide of the present invention can be used to treat any of these symptoms or diseases.

Regeneration

A therapeutic polypeptide of the present invention can be used to differentiate, proliferate, and attract cells, fostering to the regeneration of tissues. (See, Science 276:59-87 (1997).) The regeneration of tissues could be used to repair, replace, or protect tissue damaged by congenital defects, trauma (wounds, burns, incisions, or ulcers), age, disease (e.g. osteoporosis, osteoarthritis, periodontal disease), surgery, including cosmetic plastic surgery, fibrosis, reperfusion injury, or systemic cytokine damage.

Tissues that could be regenerated with the contribution of a therapeutic protein of the invention include organs (e.g. pancreas, intestine, kidney, skin, endothelium), vascular (including vascular endothelium), nervous, hematopoietic, and skeletal (bone, cartilage, tendon, and ligament) tissue. Preferably, regeneration incurs a small amount of scarring, or occurs without scarring. Regeneration also may include angiogenesis.

Moreover, a therapeutic polypeptide of the present invention may increase regeneration of tissues difficult to heal. For example, increased tendon/ligament regeneration would quicken recovery time after damage. A therapeutic polypeptide of the present invention could also be used prophylactically in an effort to avoid damage. Specific diseases that could be treated include tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using a therapeutic polypeptide of the present invention to proliferate and differentiate nerve cells. Diseases that could be treated using this method include central and peripheral nervous system diseases, neuropathies, or mechanical and traumatic disorders (e.g. spinal cord disorders, head trauma, cerebrovascular disease, and stoke). Specifically, diseases associated with peripheral nerve injuries, peripheral neuropathy, localized neuropathies, and central nervous system diseases (e.g. Alzheimer's disease, Parkinson's disease, Huntington's disease, amyotrophic lateral sclerosis, and Shy-Drager syndrome), could all be treated using therapeutic proteins of the present invention. With respect to CNS disorders, numerous means are known in the art for facilitating therapeutic access to brain tissue, including methods for disrupting the blood brain barrier, and methods of coupling therapeutic agents to moieties that provide for transport into the CNS. In one embodiment, a therapeutic nucleic acid is engineered so as to encode a fusion protein, which fusion protein comprises a transport moiety and a therapeutic protein.

Chemotaxis

In one embodiment, a therapeutic polypeptide of the present invention possesses a chemotaxis activity. A chemotaxic molecule attracts or mobilizes cells (e.g. monocytes, fibroblasts, neutrophils, T-cells, mast cells, eosinophils, epithelial and/or endothelial cells) to a particular site in the body, such as inflammation or infection. The mobilized cells can then fight off and/or heal the particular trauma or abnormality.

A therapeutic polypeptide of the present invention may increase chemotaxic activity of particular cells. These chemotactic molecules can then be used to treat inflammation, infection, or any immune system disorder by increasing the number of cells targeted to a particular location in the body. For example, chemotaxic molecules can be used to treat wounds and other trauma to tissues by attracting immune cells to the injured location. Chemotactic molecules of the present invention can also attract fibroblasts, which can be used to treat wounds.

It is also contemplated that a therapeutic polypeptide of the present invention may inhibit chemotactic activity. These molecules could also be used to treat disorders. Thus, a therapeutic polypeptide of the present invention could be used as an inhibitor of chemotaxis.

Especially preferred for use are protherapeutic proteins that are activated in the vicinity of target tissues.

Additional therapeutic polypeptides contemplated for use include, but are not limited to, growth factors (e.g., growth hormone, insulin-like growth factor-1, platelet-derived growth factor, epidermal growth factor, acidic and basic fibroblast growth factors, transforming growth factor-β, etc.), to treat growth disorders or wasting syndromes; and antibodies (e.g., human or humanized), to provide passive immunization or protection of a subject against foreign antigens or pathogens (e.g., H. Pylori), or to provide treatment of arthritis or cardiovascular disease; cytokines, interferons (e.g., interferon (INF), INF-α2b and 2a, INF-αN1, INF-β1b, INF-gamma), interleukins (e.g., IL-1 to IL-10), tumor necrosis factor (TNF-α TNF-β), chemokines, granulocyte macrophage colony stimulating factor (GM-CSF), polypeptide hormones, antimicrobial polypeptides (e.g., antibacterial, antifungal, antiviral, and/or antiparasitic polypeptides), enzymes (e.g., adenosine deaminase), gonadotrophins, chemotactins, lipid-binding proteins, filgastim (Neupogen), hemoglobin, erythropoietin, insulinotropin, imiglucerase, sarbramostim, tissue plasminogen activator (tPA), urokinase, streptokinase, phenylalanine ammonia lyase, brain-derived neurotrophic factor (BDNF), nerve growth factor (NGF), thrombopoietin (TPO), superoxide dismutase (SOD), adenosine deamidase, catalase calcitonin, endothelian, L-asparaginase pepsin, uricase trypsin, chymotrypsin elastase, carboxypeptidase lactase, sucrase intrinsic factor, calcitonin parathyroid hormone (PTH)-like, hormone, soluble CD4, and antibodies and/or antigen-binding fragments (e.g, FAbs) thereof (e.g., orthoclone OKT-e (anti-CD3), GPIIb/IIa monoclonal antibody).

Vaccination

In one embodiment, the invention provides methods for vaccinating a patient. The methods comprise administering a composition of the invention capable of producing the desired epitope. In a preferred embodiment, the composition comprises a therapeutic nucleic acid construct capable of expressing a protein comprising the epitope.

Cosmetic Applications

In one embodiment, the invention provides compositions for cosmetic use. The cosmetics comprise an chitosan-nucleic acid polyplex composition of the invention in a formulation suitable for cosmetic use.

Powdered Formulations

The chitosan-nucleic acid polyplex compositions of the invention include powders. In a preferred embodiment, the invention provides a dry powder chitosan-nucleic acid polyplex composition. In a preferred embodiment, the dry powder chitosan-nucleic acid polyplex composition is produced through the dehydration of a chitosan-nucleic acid polyplex dispersion of the invention. Dehydration methods include but are not limited to lyophilization and spray drying.

In one embodiment, a concentrated dispersion is dehydrated and then subsequently pH adjusted upon rehydration as needed. For example, in one embodiment, a concentrated dispersion having a pH greater than 4.5 is first dehydrated, and then pH adjusted to between 3.5-4.5 upon rehydration. In another embodiment, the pH adjustment is not required, and the rehydrated composition has a pH below 4.5.

Pharmaceutical Formulations

The present invention also provides “pharmaceutically acceptable” or “physiologically acceptable” formulations comprising highly acidic chitosan-nucleic acid polyplex compositions of the invention. Such formulations can be administered in vivo to a subject in order to practice treatment methods.

As used herein, the terms “pharmaceutically acceptable” and “physiologically acceptable” refer to carriers, diluents, excipients and the like that can be administered to a subject, preferably without producing excessive adverse side-effects (e.g., nausea, abdominal pain, headaches, etc.). Such preparations for administration include sterile aqueous or non-aqueous solutions, suspensions, and emulsions.

Pharmaceutical formulations can include carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, anti-oxidants, chelating agents, and inert gases and the like.

A pharmaceutical formulation can be formulated to be compatible with its intended route of administration. The subject compositions are well suited to the transfection of mucosal epithelial tissues. In a preferred embodiment, pharmaceutical compositions of the invention are of a formulation suitable for administration to mucosal epithelial tissue.

For oral administration, a composition can be incorporated with excipients and used in the form of tablets, troches, or capsules, e.g., gelatin capsules. Pharmaceutically compatible binding agents, and/or adjuvant materials can be included in oral formulations. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or flavoring.

Formulations can also include carriers to protect the composition against rapid degradation or elimination from the body, such as a controlled release formulation, including implants and microencapsulated delivery systems. For example, a time delay material such as glyceryl monostearate or glyceryl stearate alone, or in combination with a wax, may be employed.

Suppositories and other rectally administrable formulations (e.g., those administrable by enema) are also contemplated. Further regarding rectal delivery, see, for example, Song et al., Mucosal drug delivery: membranes, methodologies, and applications, Crit. Rev. Ther. Drug. Carrier Syst., 21:195-256, 2004; Wearley, Recent progress in protein and peptide delivery by noninvasive routes, Crit. Rev. Ther. Drug. Carrier Syst., 8:331-394, 1991.

Additional pharmaceutical formulations appropriate for administration are known in the art and are applicable in the methods and compositions of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)).

Administration

Any of a number of administration routes are possible and the choice of a particular route will in part depend on the target tissue. Administration to epithelial tissue is preferred. Especially preferred is administration to epithelial tissue selected from the group consisting of gastrointestinal tract, respiratory tract, lung, sinus cavity, oral cavity, urinary tract, bladder, vaginal, uterine, cervical, eye, esophagus, salivary gland, nasolaryngeal tissue, kidneys, larynx/pharynx, and skin.

Syringes, endoscopes, cannulas, intubation tubes, enema kits, catheters, nebulizers, inhalers and other articles may be used for administration.

The doses or “effective amount” for treating a subject are preferably sufficient to ameliorate one, several or all of the symptoms of the condition, to a measurable or detectable extent, although preventing or inhibiting a progression or worsening of the disorder or condition, or a symptom, is a satisfactory outcome. Thus, in the case of a condition or disorder treatable by expressing a therapeutic nucleic acid in target tissue, the amount of therapeutic protein produced to ameliorate a condition treatable by a method of the invention will depend on the condition and the desired outcome and can be readily ascertained by the skilled artisan. Appropriate amounts will depend upon the condition treated, the therapeutic effect desired, as well as the individual subject (e.g., the bioavailability within the subject, gender, age, etc.). The effective amount can be ascertained by measuring relevant physiological effects.

Veterinary applications are also contemplated by the present invention. Accordingly, in one embodiment, the invention provides methods of treating non-human mammals, which involve administering a composition of the invention to a non-human mammal in need of treatment.

Oral Administration

The compounds of the invention may be administered orally. Oral administration may involve swallowing, so that the compound enters the gastrointestinal tract. Compositions of the invention may also be administered directly to the gastrointestinal tract.

Formulations suitable for oral administration include solid formulations such as tablets, capsules containing particulates, liquids, or powders, lozenges (including liquid-filled), chews, multi- and nano-particulates, gels, films, ovules, and sprays.

Liquid formulations include suspensions, solutions, syrups and elixirs. Liquid formulations may be prepared by the reconstitution of a solid.

Tablet dosage forms generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a film-forming polymer, a binder, a solvent, a humectant, a plasticiser, a stabiliser or emulsifier, a viscosity-modifying agent and a solvent. Some components of the formulation may perform more than one function.

Also included in the invention are multiparticulate beads comprising a composition of the invention.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Other suitable release technologies such as high energy dispersions and osmotic and coated particles are known.

Parenteral Administration

Suitable means for parenteral administration include intravenous, intraarterial, intraperitoneal, intrathecal, intraventricular, intraurethral, intrasternal, intracranial, and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents, but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by sterile filtration, may readily be accomplished using standard pharmaceutical techniques well known to those skilled in the art.

The solubility of compounds used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound.

Topical Administration

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions.

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

Inhaled/Intranasal Administration

The compounds of the invention can also be administered intranasally or by inhalation, typically in the form of a dry powder (either alone, as a mixture, for example, in a dry blend with lactose, or as a mixed component particle) from a dry powder inhaler or as an aerosol spray from a pressurised container, pump, spray, atomiser, or nebuliser, with or without the use of a suitable propellant.

Capsules, blisters and cartridges for use in an inhaler or insufflator may be formulated to contain a powder mix of the compound of the invention, a suitable powder base such as lactose or starch and a performance modifier such as l-leucine, mannitol, or magnesium stearate.

Formulations for inhaled/intranasal administration may be formulated to be immediate and/or modified release. Modified release formulations include delayed-, sustained-, pulsed-, controlled-, targeted and programmed release.

Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

Ocular/Aural Administration

The compounds of the invention may also be administered directly to the eye or ear, typically in the form of drops. Other formulations suitable for ocular and aural administration include ointments, biodegradable (e.g. absorbable gel sponges, collagen) and non-biodegradable (e.g. silicone) implants, wafers, lenses and particulate systems. Formulations may also be delivered by iontophoresis.

Formulations for ocular/aural administration may be formulated to be immediate and/or modified release. Modified release formulations include delayed-, sustained-, pulsed-, controlled-, targeted, or programmed release.

### EXPERIMENTAL

TABLE 1
Materials and Equipment

| Material and Equipment | Supplier |
|---|---|
| Chitosan (23 mer, 98% DDA) | Biosyntech |
| pDNA (pCHS4-3xFLAG-CMV-SEAP- | enGene Inc. |
| attB) | |
| pDNA (pCMV-INT) | enGene Inc. |
| pDNA (gWIZ-SEAP) | Aldevron LLC |
| pDNA (gWIZ-Luciferase) | Aldevron LLC |
| Syringe filters 25-mm, 0.2 μm Supor | Pall |
| membrane | |
| Syringe filters 32-mm, 0.2 μm Supor | Pall |
| membrane | |
| Disposable cuvettes, PS, 1.5 mL semi- | Plastibrand |
| micro | |
| Folded capillary Zeta cells | Malvern Instruments |
| TFF cartridge, 73 cm2, 1 mm ID, 100K | GE Healthcare |
| MWCO | |
| TFF cartridge, 850 cm2, 1 mm ID, 100K | GE Healthcare |
| MWCO | |
| TFF cartridge, 73 cm2, 1 mm ID, 500K | GE Healthcare |
| MWCO | |
| Syringe pump, NE-1000 | New Era Pump Systems |
| | Inc. |
| Syringe pump, NE-1000 | New Era Pump Systems |
| | Inc. |
| L/S Digistaltic Pump System | Masterflex |
| L/S Pumpheads (for performance | Masterflex |
| tubing) | |
| L/S Pumpheads (for high-performance | Masterflex |
| tubing) | |
| L/S Pump | Masterflex |
| I/P Pump | Masterflex |
| I/P Pumphead | Masterflex |
| Particle Sizer, Zetasizer Nano (ZEN | Malvern Instruments |
| 3600) | |
| pH Meter, Accumet AB15 | Fisher Scientific |
| pH Meter, ISFET probe | IQ Scientific |
| UV Spectrophotometer, Ultrospec 2100 | Biochrom Ltd. |
| pro | |
| Qubit Fluorometer, cat # Q32857 | Invitrogen |
| FluorChem Imaging System including | Alpha Inotech Corp |
| AlphaEaseFC software v3.1 | |
| Luminometer (LmaxII384) including | Molecular Devices |
| Softmax Pro software v4.7.1 | |
| Small-scale TFF System (MidGee) | GE Healthcare |
| Mid-scale TFF System (FlexStand) | GE-Healthcare |

For additional description of materials and methods for inline mixing and concentrating polyplex compositions, see WO 2009/039657, which is expressly incorporated herein in its entirety by reference.

Polyplex Formulation Naming Convention.

C(23,98)-N20-Ac31-pH4.8-c150-Suc9%-Pbn0.1%

| C(23, 98) | N20 | Ac31 | pH 4.8 | c150 | Suc9% | Pbn0.1% |
|---|---|---|---|---|---|---|
| Chitosan | NP ratio = | AcOH = | pH = | DNA = | Sucrose = | Parabens = |
| (23 mer, 98% DDA) | 20 | 31 mM | 4.8 | 150 ug/mL | 9% w/w | 0.1% w/w |

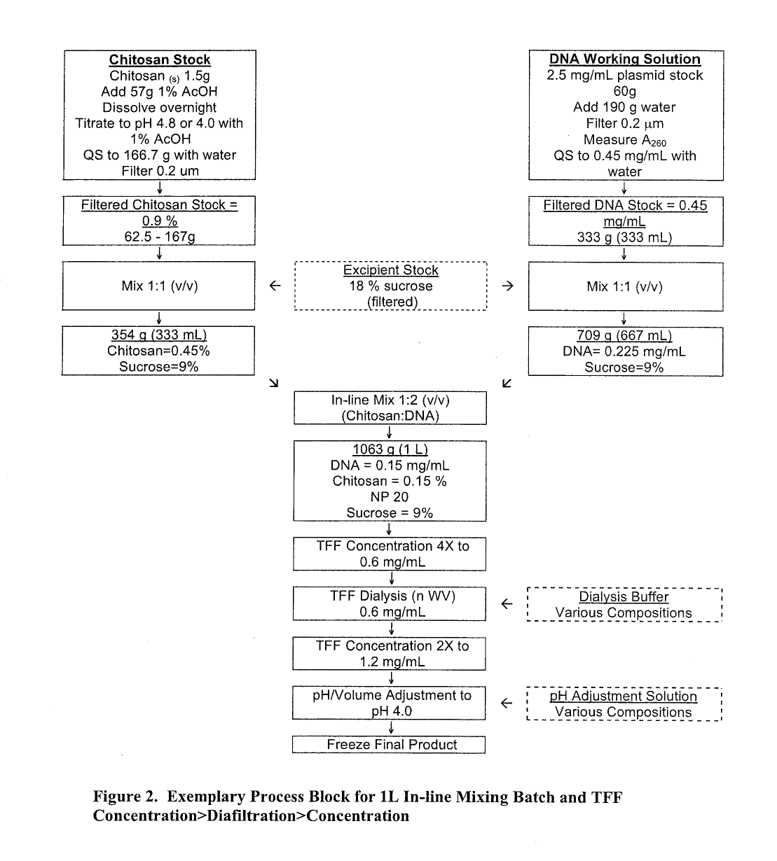

A typical process block for manufacturing a 1 L batch followed by TFF concentration is shown in FIG. 2.

Small-Scale In-Line Mixing

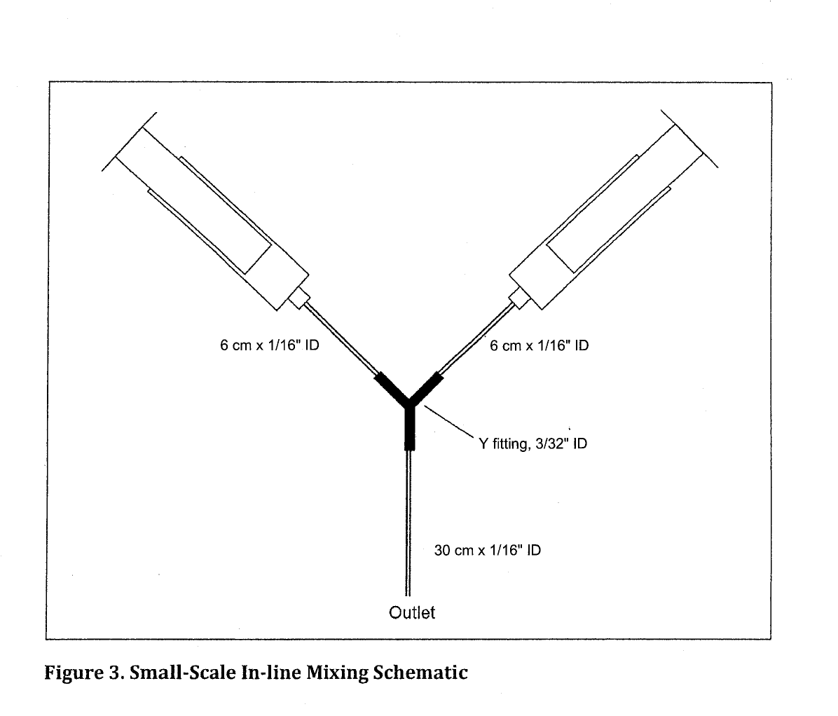

A simple small-scale in-line mixing apparatus was tested using syringe pumps, 1/16-inch ID silicone tubing; and a 3/32-inch ID polypropylene junction in a Y configuration. A schematic of the set-up with 3 mL capacity syringes and a Y-junction is shown in FIG. 3. Note that the maximum syringe volume for this set-up is 60 mL. This process was used to make polyplexes with final DNA concentration of 150 μg/mL at an NP ratio of 20 using 24mer/98% DDA chitosan. DNA and chitosan feedstocks were mixed at a volume ratio of 2:1 to produce homogeneous polyplex formulations.

Mid-Scale In-Line Mixing

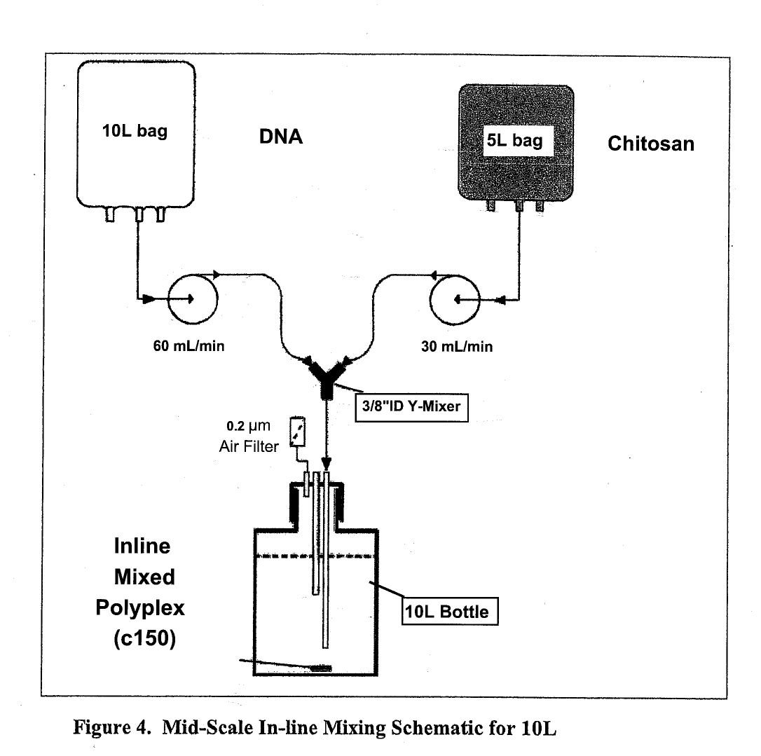

A simple mid-scale in-line mixing apparatus was tested using peristaltic pumps, 3/16-inch ID silicone tubing; and a 3/16-inch ID polypropylene junction in a Y configuration. A schematic of the set-up with a Y-junction is shown in FIG. 4. Note that the maximum output volume for this set-up limited only by the volume of the feedstock vessels. This process was used to make polyplexes with final DNA concentration of 150 μg/mL at an NP ratio of 20 using 24mer/98% DDA chitosan. DNA and chitosan feedstocks were mixed at a volume ratio of 2:1 to produce homogeneous polyplex formulations.

TFF Process (Concentration)

Prior to carrying out TFF studies, the hollow fiber filters were rinsed and cleaned according to the manufacturer's instructions.

TFF Concentration #1

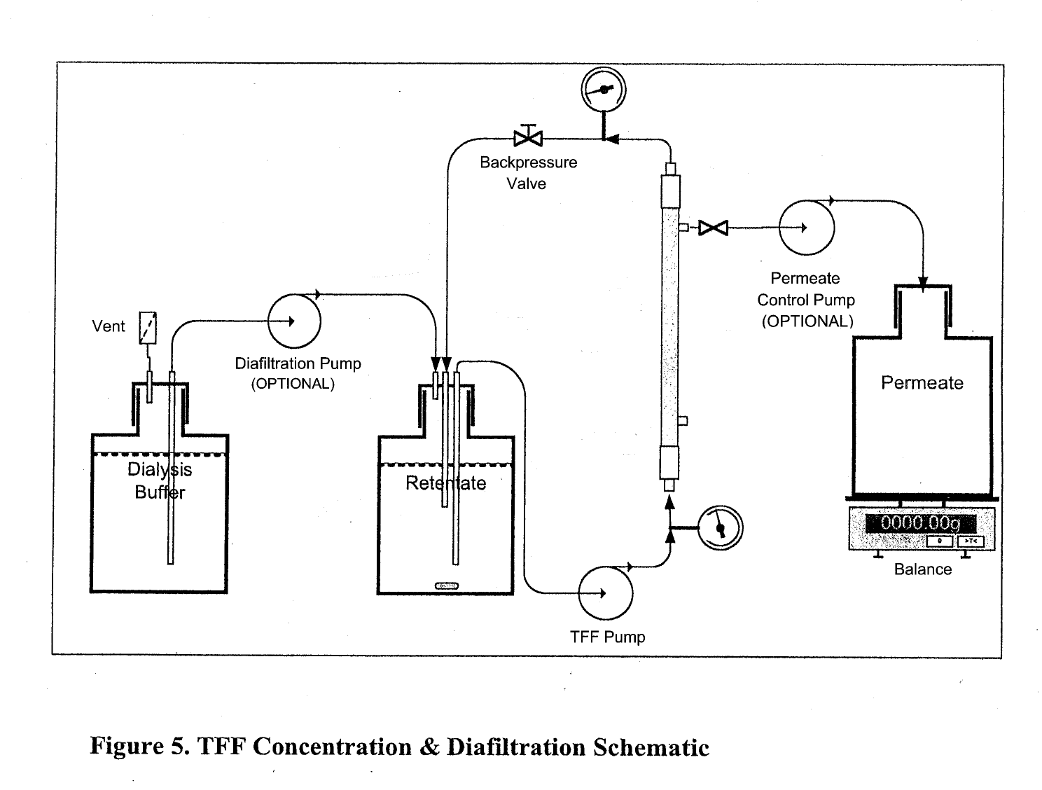

To carry out concentration, the TFF system was set up as shown in the schematic diagram (FIG. 5) and purged of residual water. After closing the permeate valve and fully opening the backpressure valve, the DNA-chitosan polyplex was added to the product reservoir. Concentration was started by switching on the pump, fully opening the permeate valve (and starting the optional pump) and then adjusting the backpressure valve to the target filter inlet pressure. During the concentration process, the mass of permeate collected was monitored on a balance and used to determine when the target DNA concentration had been achieved. After the target volume reduction was attained, the concentration process was stopped by closing the permeate valve and fully opening the backpressure valve. See equation below:

[DNA]_{Retentate}=[DNA]_{Initial}×(Mass_{Initial}/(Mass_{Initial}−Mass_{Permeate}))

TFF Diafiltration

In some batches, a diafiltration step (buffer exchange) was inserted in the concentration process. For example, starting from 0.15 mg/mL of DNA, the polyplex is concentrated to 0.60 mg/mL, then diafiltered for a certain number of wash volumes while maintaining a 0.60 mg/mL concentration, then further concentrated to 1.20 mg/mL.

To carry out diafiltration, the permeate outlet line was changed to a new tarred collection vessel, and then the buffer line was connected to the retentate vessel via the vent port. This creates a sealed system with no atmospheric venting. Next, the permeate valve was opened and/or the permeate pump was started (same flow rate as above). This creates a vacuum in the retentate vessel as permeate is withdrawn that in turn draws dialysis buffer into the retentate vessel. In this manner, the retentate fluid is maintained at a constant level by being continuously replenished as permeate is discharged. This is the dialysis process. In some cases (when insufficient vacuum resulted due to atmospheric leaks in the system), dialysis buffer was pumped into the retentate at the same rate as the permeate. Diafiltration was carried out for a target number of wash volumes (1 wash volume=the volume of retentate). To stop dialysis, the permeate was closed (valve and stop the permeate pump), and the retentate vessel was opened to the atmosphere and closed to the buffer line.

TFF Concentration #2

After diafiltration, TFF concentration was resumed. After the target volume reduction was attained, the concentration process was stopped by closing the permeate valve and fully opening the backpressure valve. After purging the retentate fluid lines and collecting the final product, a sample of this post-TEE product was submitted for analytical testing and DNA concentration by the picogreen assay. The remainder was either stored immediately at −80° C., or stored at 4° C. until completion of analytical testing, and then either promptly used or frozen for storage.

Post-TFF pH Adjustment

Unless otherwise described, the pH of the final post-TFF product was promptly adjusted for pH by the addition of a pH adjustment buffer. The buffer compositions were generally comprised of acetic acid and/or chitosan in a solution of sucrose. This solution was added to the final TFF product at a volume ratio of 4.5:95.5, respectively. This additional volume would reduce the concentration of the post-TFF product by 4.5%.

Analytical Testing

Particle Sizing

Particle size measurements were made using a Zetasizer Nano light scattering instrument.

Except where noted, samples were diluted 20-fold in 10 mM NaCl (0.4 mL minimum) and loaded into a disposable cuvette. The Zetasizer was programmed to incubate the sample for 3 minutes at 25° C. prior to triplicate 3-minute measurements. Z-average and polydispersity (PDI) were reported with standard deviation (n=3). For diluted samples, the Zetasizer was programmed to use viscosity and refractive index of 10 mM NaCl.

Zeta Potential

Zeta potential measurements were made using a Zetasizer Nano light scattering instrument. In general, undiluted samples were loaded into a Zetasizer folded capillary cell (0.8 mL minimum). The Zetasizer was programmed to incubate the sample for 3 minutes at 25° C. prior to replicate measurements (number of replicates were automatically determined by Zetasizer software). Zeta potential values were reported with standard deviation (n=3). The Zetasizer was also programmed to account for the final composition of the samples with regards to viscosity and dielectric constant.

Short-Term Stability by Freezing

For short-term stability studies, final polyplex product was frozen and stored at the appropriate temperature (−20° C., −30° C. or −80° C.) overnight. In some cases, samples were rapidly frozen in dry ice/ethanol baths, then stored at the appropriate temperatures. At the appropriate times, samples were thawed to room temperature and analyzed as described.

Chitosanase Digestion

50 ul of polyplex were digested with 50 uL of 4.44 U/mL chitosanase for 2 h at 37° C. (Stock chitosanase concentration is 62 U/mL and was diluted with cold 50 mM NaOAc, pH 5.5 at 37° C.) For C(24,98)-N40-c75 particles, it's best to digest 0.909-1.818 mM chitosan to release all the DNA, so the particles were diluted 1/10 and 1/5 in 150 mM NaOAc, pH 5.5 at 37° C.

DNA Quantification with PicoGreen

Prior to DNA measurement using the PicoGreen assay, total DNA must be released from the polyplex by chitosanase. Following release, DNA is subjected to DNA digest with a suitable restriction enzyme to linearize the supercoiled DNA plasmids.

EcoR1 Digestion

After incubation, X uL of the chitosanase-digested sample was added to 5 uL of EcoR1 and 5 uL of EcoR1 buffer and brought to a final 50 uL final volume with MilliQ water. (Sample volume X uL was adjusted so that final DNA concentration was 4 ng/uL.) The EcoR1 sample was then incubated for 30 min at 37° C.

PicoGreen Assay

The PicoGreen Quant-iT ds DNA HS Assay kit was supplied with two buffers (A and B) and two standards (1 and 2). Buffer A was diluted 1:20 into Buffer B to make solution “A/B”. Standards 1 and 2 were diluted 20-fold with solution A/B (10 uL into 200 uL). Final concentrations for standards 1 and 2 were 0 and 10 ng/uL, respectively.

10 to 20 uL of EcoR1 digested sample was brought to a final volume of 200 uL with solution A/B, briefly vortexed, incubated at RT for 2 minutes and then measured for fluorescence on the Qubit Fluorometer according to manufacturer instructions.

Gel Electrophoresis

For verification of DNA capture into the polyplex, samples were subjected to gel electrophoresis. Samples aliquots of 1-5 uL (target of 800 ng DNA) were combined with 2 uL of TrackIt loading buffer and brought to a final 10 uL volume with water. Standard lanes were loaded with Supercoiled DNA ladder. The samples were resolved on a 0.8% agarose gel containing ethidium bromide (50 ug/mL) at 120 V for 45 minutes. The gel was imaged with the Fluor Chem Imaging System.

SEAP Assay

The SEAP assay was performed using the SEAP Chemiluminescent Assay kit. All reagents for the assay were equilibrated at 25° C. for 30 min before use. Standards for the assay were prepared by dissolving placental alkaline phosphatase to 1 mg/mL in 1× dilution buffer from the kit spiked with 0.1% bovine serum albumin and 50% glycerol and then diluting by 10-fold serial dilutions with DMEM to 0.01 pg/uL. Standards and thawed samples were then diluted 1 in 4 with dilution buffer, heat inactivated at 65° C. for 30 min, incubated on ice for 2 min, centrifuged (16100×rcf for 2 min at RT) and the supernatants transferred to new tubes. After equilibrating at 25° C. for 5 min, 50 uL of the samples and standards were added to each well of a Microlite-1 plate in duplicate. Inactivation buffer (50 uL) was then added to each well and pipetted up and down gently to mix, without creating bubbles and incubated for 5 min. The substrate/enhancer reagent was prepared during the 5 min incubation at a ratio for 1:19 of substrate to enhancer. The substrate/enhancer was then added to each well, incubated for 20 min and then the plate was read in the luminometer with an integration time of 1 sec.

Ninhydrin Assay (Total Chitosan)

The total chitosan concentration in polyplexes was determined using the ninhydrin assay. Briefly, polyplexes are diluted to contain 1-2 mM glucosamine with sodium acetate at a final concentration of 150 mM, pH 5.5. A standard curve prepared from chitosan of the same chain length is diluted with 70 mM sodium acetate, pH 5.5 to concentrations of 0.5-7.5 mM glucosamine. Diluted polyplexes and standards are then digested for 2 h at 37° C. with an equal volume of 5 U/ml chitosanase in 50 mM sodium acetate, pH 5.5. After the 2 h incubation, 100 ul of the digested polyplexes and standards are then added to glass tubes containing 400 ul of 70 mM sodium acetate, pH 5.5. Ninhydrin reagent (250 uL) is then added to each sample, the tubes then vortexed briefly and boiled for 10 min. After cooling at room temperature for 15 min, 1.25 ml of ethanol is added and the absorbance values measured at 550 nm. The chitosan concentrations in the polyplexes are calculated from the slope and y-intercept of the linear standard curve and adjusted with the initial dilution factor.

### Example 1

### First Small-Scale Trial for Diafiltration

Table 2 describes the batch parameters for a test of diafiltration. A c150-pH4.0 formulation was used as the starting feedstock. A pH/acetate adjustment step was added as the final step before fill & finish.

TABLE 2
Parameters and results.

| Nominal starting formulation: | | C(23, 98)-N20-Ac31-pH4.0-c150 | | | |
|---|---|---|---|---|---|
| Batch size: | | 92 g | | | |
| Process: | | TFF concentration #1 (4-fold to c600) | | | |
| | | TFF diafiltration 6WV | | | |
| | | TFF concentration #2 (2.2-fold to c1300) | | | |
| | | pH adjustment to c1250 | | | |
| | | Fill/Finish −80° C. | | | |
| Dialysis Buffer | | 10 mM HAc, 9.3% sucrose | | | |
| Buffer adjustment solution | | 2.24%C(23, 98)-Ac72-pH3.3 | | | |
| | | | Zeta | | |
| | Diameter | | Potential | | Osmolality |
| | (nm) | PDI | (mV) | pH | (mmol/kg) |
| Pre-TFF | 103 | 0.17 | n.d. | 4.6 | n.d. |
| Post-TFF #1 | n.d. | n.d. | n.d. | n.d. | n.d. |
| Post-Diafiltration | n.d. | n.d. | n.d. | n.d. | n.d. |
| Post-TFF #2 | 106 ¹ | 0.17 ¹ | n.d. | n.d. | n.d. |
| Post-Adjustment | 106 ² | 0.17 ² | n.d. | 3.8 | n.d. |
| Freeze/Thaw | 106 | 0.17 | +30 | 3.8 | 344 |

¹ Measured after 17 h at RT
² Measured after 6 h at RT

### Example 2

### Second Small-Scale Trial for Diafiltration

A second test of diafiltration (Table 3) was a 3-fold larger batch size and utilized a c150-pH4.0 formulation as the starting feedstock. A pH/acetate adjustment step was added as the final step before fill & finish.

TABLE 3
Parameters and results.

| | |
|---|---|
| Nominal starting formulation: | C(23,98)-N20-Ac30-pH4.0-c150 |
| Batch size: | 302 g |
| Process: | TFF concentration #1 (4-fold to c600) |
| | TFF diafiltration 6WV |
| | TFF concentration #2 (2-fold to c1200) |
| | pH adjustment to c1150 |
| | Fill/Finish −80 C. |
| Dialysis Buffer | 10 mM HAc, 9.3% sucrose |
| Buffer adjustment solution | 2.24%C(23,98)-Ac72-pH3.3 |

| | Diameter (nm) | PDI | pH |
|---|---|---|---|
| Pre-TFF | 108 | 0.165 | n.d. |
| Post-TFF #1 | 108 | 0.168 | n.d. |
| Post-Diafiltration | n.d. | n.d. | n.d. |
| Post-TFF #2 | 111 | 0.174 | n.d. |
| Post-Adjustment | 111 | 0.185 | 3.9 |
| Freeze/Thaw | 116 | 0.207 | n.d. |

### Example 3

### Small-Scale Batch

A third test of diafiltration (Table 4) was carried out. This batch also utilized a c150-pH4.0 formulation as the starting feedstock.

TABLE 4
Parameters and results.

| Nominal starting formulation: | | C(23, 98)-N20-Ac30-pH4.0-c150 | | | |
|---|---|---|---|---|---|
| Batch size: | | 90 g | | | |
| Process: | | TFF concentration #1 (4-fold to c600) | | | |
| | | TFF diafiltration 6.1 WV | | | |
| | | TFF concentration #2 (2-fold to c1200) | | | |
| | | pH adjustment to c1150 | | | |
| | | Fill/Finish −80° C. | | | |
| Dialysis Buffer | | 10 mM HAc, 9.3% sucrose, pH 3.25 | | | |
| Buffer adjustment solution | | 2.2%C(23, 98)-Ac72-pH3.3 | | | |
| TFF Flow Rate & Shear | | 65-70 mL/min & ~5000 s⁻¹ | | | |
| TFF Permeate Flux | | Pump controlled ~3.5 g/min | | | |
| | | | Zeta | | |
| | Diameter | | Potential | | Osmolality |
| | (nm) | PDI | (mV) | pH | (mmol/kg) |
| Pre-TFF | 94.5 | 0.16 | n.d. | 4.12 | n.d. |
| Post-TFF #1 | n.d. | n.d. | n.d. | n.d. | n.d. |
| Post-Diafiltration | n.d. | n.d. | n.d. | n.d. | n.d. |
| Post-TFF #2 | 96.3 | 0.14 | n.d. | 4.02 | n.d. |
| Post-Adjustment | 96.2 | 0.14 | n.d. | 3.62 | n.d. |
| Freeze/Thaw | 97.3 | 0.15 | +42 | n.d. | 0.59 |

### Example 4

### Mid-Scale Batch

TABLE 5
Parameters and results.

| Nominal starting formulation: | | C(23, 98)-N20-Ac31-pH4.8-c150 | | | |
|---|---|---|---|---|---|
| Plasmid(s) | | gWiZ SEAP | | | |
| Batch size: | | 3.6 kg | | | |
| TFF Cartridge Surface Area | | 0.085 m² | | | |
| TFF Volume/Surface Ratio | | 42.3 kg/m² | | | |
| Process: | | TFF concentration #1 (4-fold to c600) | | | |
| | | TFF diafiltration 6WV | | | |
| | | TFF concentration #2 (2-fold to c1200) | | | |
| | | pH adjustment to c1150 | | | |
| | | Fill/Finish −80° C. | | | |
| Dialysis Buffer | | 10 mM HAc, 9.3% Sucrose | | | |
| Buffer adjustment solution | | 7.5 mM chitosan; 48 mM HAc | | | |
| TFF Flow Rate & Shear | | 1000 mL/min & 3300 s⁻¹ | | | |
| TFF Permeate Flux | | Pump controlled ~35 g/min | | | |
| | | | Zeta | | |
| | Diameter | | Potential | | Osmolality |
| | (nm) | PDI | (mV) | pH | (mmol/kg) |
| Pre-TFF | 88 | 0.154 | 37 | 4.15 | 290 |
| Post-TFF #1 | 90 | 0.130 | 36 | n.d. | n.d. |
| Post-Diafiltration | 92 | 0.136 | 42 | n.d. | n.d. |
| Post-TFF #2 | 93 | 0.147 | 42 | n.d. | n.d. |
| Post-Adjustment | 93 | 0.143 | 44 | 3.86 | n.d. |

pH Shift during TFF Concentration Step(s)

It has been noted in several batches that utilized the TFF concentration process, that pH generally shifts 0.2 to 0.5 units upward. This is due to changes in the relative concentrations of total acetate versus chitosan in the formulation as TFF proceeds; i.e. pH is a function of [Chitosan]/[Acetate]. This was modeled. pH was monitored closely after the diafiltration step as DNA was increased from 0.60 mg/mL to 2.0 mg/mL. For the model, the following assumptions were made:

Assume that [Acetate] is constant after dialysis at 10 mM

Assume arbitrary [Chitosan] of 1 mM at start of concentration step

Assume [Chitosan] increases proportionally with volume reduction

Assume that formulation pH adheres to the Henderson-Hasselbach buffer theory

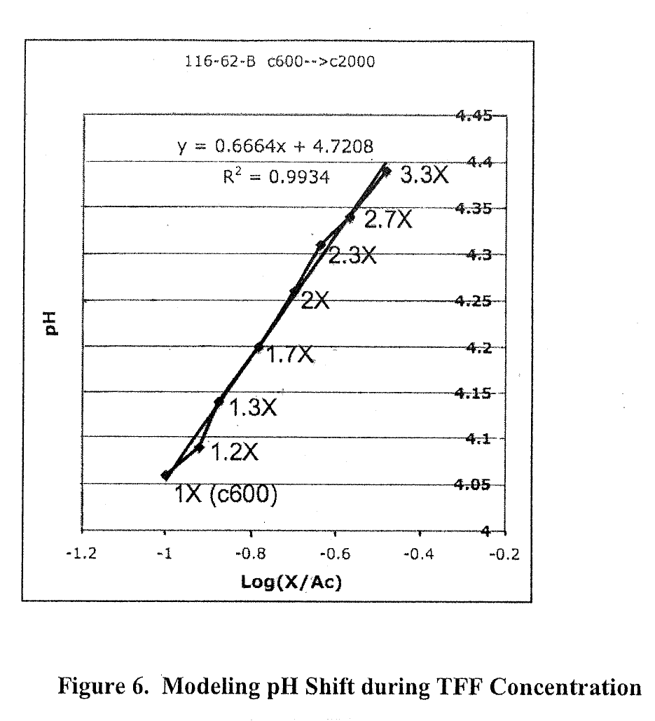

To model this pH shift, pH versus Log(^{[Chitosan]}/_{[Acetate]}) was plotted (FIG. 6) and the resulting curve was determined:

pH=0.6664×Log(^{[Chitosan]}/_{[Acetate]})+4.7208

FIG. 6. Modeling pH Shift during TFF Concentration. Each point indicates the relative volume-fold reduction (=increasing DNA concentration) of the polyplex. For example, the point labeled 2× is approximately c1200.

We can use this model to predict the pH of the c1200 formulation after spiking with acetate to result in 80 mM. Assume chitosan in c1200 is 2× (i.e. 2×1 mM=2 mM). Assume total acetate is 80 mM. pH=0.6664×Log( 2/80)+4.7208=3.65. The result is very close to the empirical result of 3.7.

This model also shows that in order to achieve a pH of 4.0 with a final desired acetate concentration of 80 mM, the chitosan concentration must be 6.6-fold greater than the starting amount for this batch. Consequently, if we carry out diafiltration with a chitosan-free buffer, this will remove nearly all of the free chitosan, and then the simultaneous targets for pH (4.0) and acetate (80 mM) cannot be achieved. Diafiltration is preferably performed with a chitosan-containing buffer.

Post-TFF Stability

A critical process parameter is after completion of the second TFF concentration step. Unlike the other prior steps, the polyplex is not stable after the concentrating to c1100 and must be adjusted to a lower pH within 1 hour of stopping TFF. Once the pH has been adjusted, the particles are stable at room temperature.

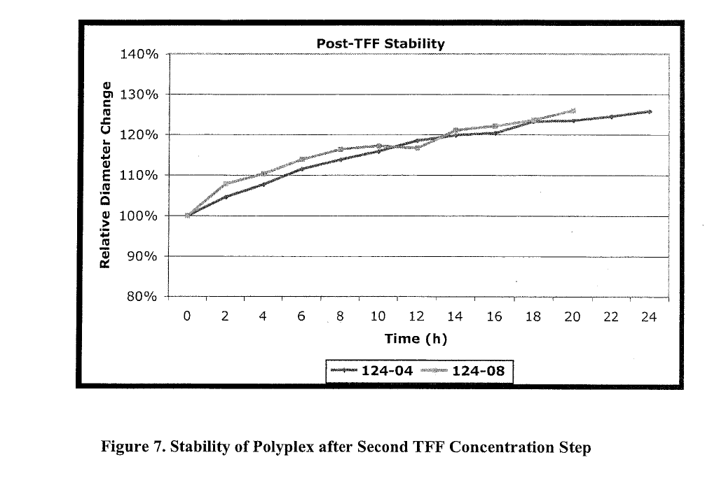

FIG. 7. Stability of Polyplex after Second TFF Concentration Step. Undiluted post-TFF sample was incubated at 25° C. and monitored for particle size every 2 hours.

### Example 5

### Mid-Scale Trial for Diafiltration with Chitosan-Containing Buffer

TABLE 6
Parameters.

| | |
|---|---|
| Nominal starting formulation: | C(23,98)-N20-Ac12-pH4.8-c150 |
| Batch size: | 1.600 kg |
| Process: | TFF concentration #1 (4-fold to c600) |
| | TFF diafiltration 4 WV |
| | TFF concentration #2 (1.8-fold to c1100) |
| | pH adjustment to c1050 |
| | Fill/Finish −80 C. |
| TFF Cartridge Surface Area | 0.0850 m² |
| TFF Volume/Surface Ratio | 18.8 kg/m² |
| TFF Flow Rate & Shear | 3000 mL/min & 9000 s⁻¹ |
| TFF Permeate Flux | Pump controlled ~35 g/min |
| Dialysis Buffer | 0.953 mM HAc, 9.5% sucrose, |
| | 1.48 mM Chitosan, pH 5.3 |
| Buffer adjustment solution | 2.24%C(23,98)-Ac72-pH3.3 |

TABLE 7
Analytical Results

| | Diameter | | Zeta Potential | |
|---|---|---|---|---|
| | (nm) | PDI | (mV) | pH |
| Pre-TFF | 82 | 0.143 | 40 | 4.8 |
| Post-TFF #1 | 83 | 0.138 | 29 | 5.18 |
| Post-Diafiltration | 88 | 0.152 | n.d. | 5.54 |
| Post-TFF #2 | 100 | 0.169 | n.d. | 5.75 |
| Post-Adjustment | 98 | 0.180 | n.d. | 3.98 |
| Freeze/Thaw RT (5 mL) | 104 | 0.184 | n.d. | n.d. |
| Freeze/Thaw RT (10 mL) | 106 | 0.185 | n.d. | n.d. |
| Freeze/Thaw RT (15 mL) | 108 | 0.191 | n.d. | n.d. |
| Freeze/Thaw RT (20 mL) | 109 | 0.199 | n.d. | n.d. |
| Freeze/Thaw 37 C. (10 mL) | 102 | 0.184 | n.d. | n.d. |

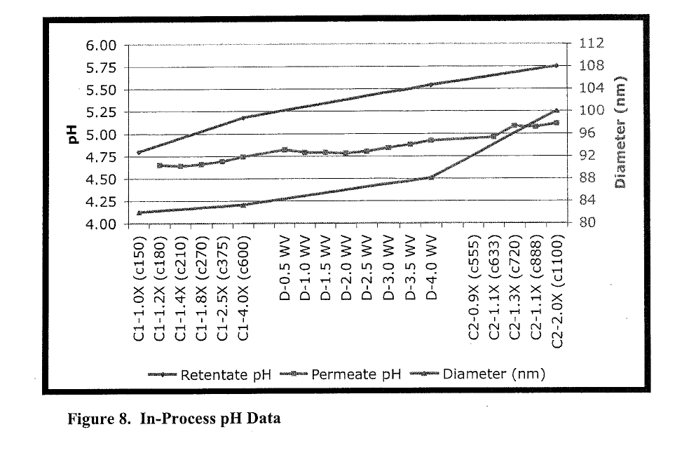

FIG. 8. In-Process pH Data. TFF fraction codes on the X-axis are as follows: C1: TFF concentration step #1; D: TFF diafiltration, indicated in # of wash volumes (WV); C2: TFF concentration step #2.

### Example 6

### Small Scale Batches with pH 4 Dialysis Buffer

To better control the pH of the product during TFF, the diafiltration buffer was modified to a lower pH. The following table summarizes the experiment and results.

TABLE 8
Parameters

| | |
|---|---|
| Nominal starting formulation: | C(23,98)-N20-Ac12-pH4.8-c150 |
| Batch size: | 0.1 kg |
| Process: | TFF concentration #1 (4-fold to c600) |
| | TFF diafiltration 4 WV |
| | TFF concentration #2 (1.8-fold to c1100) |
| | pH adjustment to c1050 |
| | Fill/Finish −80 C. |
| TFF Cartridge Surface Area | 0.0073 m² |
| TFF Volume/Surface Ratio | 13.7 kg/m² |
| TFF Flow Rate & Shear | 100 mL/min & 8000 s⁻¹ |
| TFF Permeate Flux | Pump controlled ~3 g/min |
| Dialysis Buffer | 5 mM HAc, 9.5% sucrose, 1.5 mM |
| | Chitosan, pH 4.1 |
| Buffer adjustment solution | 139 mM Chitosan, 1500 mM |
| | Acetic Acid |

TABLE 9
Analytical Results

| | Diameter | | Zeta Potential | |
|---|---|---|---|---|
| | (nm) | PDI | (mV) | pH |
| Pre-TFF | 96 | 0.16 | 35 | 4.8 |
| Post-TFF #1 | n.d. | n.d. | n.d. | n.d. |
| Post-Diafiltration | n.d. | n.d. | n.d. | n.d. |
| Post-TFF #2 | 99 | 0.16 | 33 | n.d. |
| Post-Adjustment | 99 | 0.16 | 34 | 4.0 |
| −80 C. Freeze/Thaw RT | 116 | 0.16 | n.d. | 4.0 |
| (10 mL) | | | | |

### Example 7

### Mid Scale Batches with pH 4 Dialysis Buffer

Three mid-scale batches were produced. The following tables summarize the experiment and results.

TABLE 10
Parameters

| | |
|---|---|
| Nominal starting formulation: | C(23,98)-N20-Ac12-pH4.8-c150 |
| Batch size: | 1.600 kg |
| Process: | TFF concentration #1 (4-fold to c600) |
| | TFF diafiltration 4 WV |
| | TFF concentration #2 (1.8-fold to c1100) |
| | pH adjustment to c1050 |
| | Fill/Finish −80 C. |
| TFF Cartridge Surface Area | 0.0850 m² |
| TFF Volume/Surface Ratio | 18.8 kg/m² |
| TFF Flow Rate & Shear | 3500 mL/min & 9000 s⁻¹ |
| TFF Permeate Flux | Pump controlled ~35 g/min |
| Dialysis Buffer | 5 mM HAc, 9.5% sucrose, 1.5 mM |
| | Chitosan, pH 4.1 |
| Buffer adjustment solution | 137 mM Chitosan, 1500 mM |
| | Acetic Acid |

TABLE 11
Analytical Results

| | Diameter | | Zeta Potential | |
|---|---|---|---|---|
| | (nm) | PDI | (mV) | pH |
| Pre-TFF | 89.7 ± 0.4 | 0.14 ± 0.01 | 35 ± 3 | 4.82 ± 0.02 |
| Post-TFF #1 | 91.5 ± 0.8 | 0.132 ± 0.003 | 30 ± 2 | 5.04 ± 0.01 |
| Post-Diafiltration | 93 ± 1 | 0.135 ± 0.005 | 30 ± 1 | 4.85 ± 0.02 |
| Post-TFF #2 | 93.5 ± 0.5 | 0.148 ± 0.002 | 25 ± 5 | 5.12 ± 0.08 |
| Post-Adjustment | 94 ± 1 | 0.15 ± 0.01 | 31.1 ± 0.1 | 3.99 ± 0.02 |
| −80 C. Freeze/Thaw RT | 110 ± 2 | 0.188 ± 0.004 | 32 ± 2 | 4.02 ± 0.03 |
| (10 mL) | | | | |

Results are averages of 3 batches.

In-line mixing of DNA and chitosan, TFF concentration, TFF diafiltration and a pH adjustment was done in order to manufacture c1000 polyplex with a final pH of 4.0. The final formulation also had a buffer capacity of 70-80 mM acetate and was physiologically isotonic. In addition, the nanoparticle dispersion was stable to −80° C. freeze and RT thaw for a period of at least 8 hrs after thawing.

### Example 8

### Long-Term Stability at −80° C.

The final product from mid-scale manufacturing after one-year storage at −80° C. was optically translucent and free of visible particulates (data not shown).

Chitosan-DNA nanoparticles from mid-scale batches were physically stable for up to one year at −80° C. Changes in particle diameter, polydispersity and derived count rate were negligible (TABLE 12). Small-scale batches were also stable for up to the shorter time period tested of four months.

TABLE 12
Stability at −80° C.: Particle Diameter, PDI, and DCR

| | | | | |
|---|---|---|---|---|
| | 127 days | 136 days | 167 Days | 345-360 Days |
| 0 days | (18 weeks) | (19 weeks) | (24 weeks) | (49-51 weeks) |

Small-Scale Batch

| 136-02 | 103 | nm | 103 | nm | n.d | n.d | n.d |
|---|---|---|---|---|---|---|---|
| | 0.18 | | 0.18 | | | | |
| | 6535 | kcps | 6535 | kcps | | | |
| 136-03 | 103 | nm | 103 | nm | n.d. | n.d | n.d |
| | 0.18 | | 0.18 | | | | |
| | 6352 | kcps | 6352 | kcps | | | |

Mid-Scale Batch

| DP-0088 | 112 | nm | n.d | 104 | nm | n.d | 111 | nm |
|---|---|---|---|---|---|---|---|---|
| | 0.19 | | | 0.17 | | | 0.18 | |
| | 6304 | kcps | | 7013 | kcps | | 6445 | kcps |

| DP-0089 | 109 | nm | n.d | n.d | 105 | nm | 108 | nm |
|---|---|---|---|---|---|---|---|---|
| | 0.19 | | | | 0.17 | | 0.16 | |
| | 5763 | kcps | | | 5784 | kcps | 5710 | kcps |

| DP-0090 | 108 | nm | n.d. | n.d. | n.d. | 107 | nm |
|---|---|---|---|---|---|---|---|
| | 0.19 | | | | | 0.19 | |
| | 5493 | kcps | | | | 5626 | kcps |

The chitosan-DNA nanoparticles from the mid-scale batches were electrically stable for up to one year at −80° C. Changes in conductivity and pH were negligible and within analytical error (TABLE 13). Zeta potential seemed to increase by 15-30% over the year, though fluctuations of 10% are considered normal for this assay (Malvern Instruments Technical Note MRK1031-01). Nevertheless, the electrical properties after one year were still within the product release specifications. The small-scale batches were stable for up to the shorter time period tested of four months.

TABLE 13
Stability at −80° C.: Zeta Potential, Conductivity and pH

| | | | | |
|---|---|---|---|---|
| | 127 days | 136 days | 167 Days | 345-360 Days |
| 0 days | (18 weeks) | (19 weeks) | (24 weeks) | (49-51 weeks) |

Small-Scale Batch

| 136-02 | 39 | mV | 39 | mV | n.d | n.d | n.d |
|---|---|---|---|---|---|---|---|
| | 1.04 | mS/cm | 1.04 | mS/cm | | | |
| | pH 4.09 | | pH 4.09 | | | | |
| 136-03 | 40 | mV | 40 | mV | n.d. | n.d | n.d |
| | 1.00 | mS/cm | 1.00 | mS/cm | | | |

| | |
|---|---|
| pH 4.00 | pH 4.00 |

Mid-Scale Batch

| | | | | | | | | |
|---|---|---|---|---|---|---|---|---|
| DP-0088 | 29 | mV | n.d | 36 | mV | n.d | 39 | mV |
| | 0.97 | mS/cm | | 0.97 | mS/cm | | 0.95 | mS/cm |

| | pH 4.03 | | | pH 4.02 | | | pH 3.92 |
|---|---|---|---|---|---|---|---|
| DP-0089 | 34 | mV | n.d | n.d | 39 | mV | n.d |
| | 0.93 | mS/cm | | | 0.94 | mS/cm | |

| | pH 4.04 | | | | pH 4.00 | | |
|---|---|---|---|---|---|---|---|
| DP-0090 | 32 | mV | n.d. | n.d | n.d | 39 | mV |
| | 0.93 | mS/cm | | | | 0.99 | mS/cm |

| | |
|---|---|
| pH 3.98 | pH 3.93 |

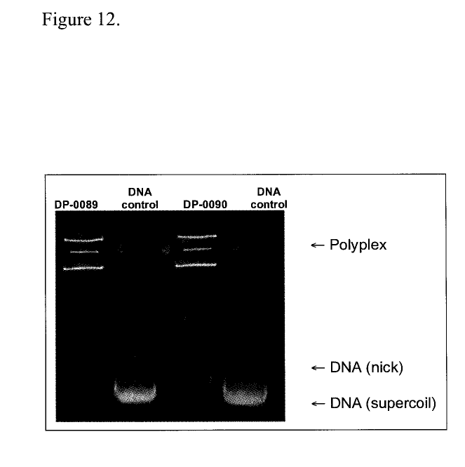

The maintained encapsulation of DNA plasmids was shown by agarose gel electrophoresis. Two mid-scale batches of polyplex after one-year storage at −80° C. were analyzed by agarose gel eletrophoresis. DNA remained encapsulated in the polyplex and was retained in the sample well and did not migrate toward the cathode (FIG. 12).

### Example 9

### Drug Product Delivery to Pig Duodenum

Drug product was delivered to the duodenum of an overnight-fasted pig via endoscopy. Briefly, a colonoscope was inserted into the anaesthetized pig's mouth, until the tip of the scope had gained entry past the pyloric sphincter into the duodenum. After IV administration of 0.3 mg/kg of Buscopan (to reduce peristalsis), the scope was further inserted 20 cm beyond the bile duct. At this point, a custom double-balloon catheter was advanced into the duodenum via the scope's delivery channel and then both distal and proximal balloons were inflated with 15 to 20 mL of saline, while ensuring that the proximal balloon was at least 5 cm distal to the bile duct. The duodenum was then washed by filling and draining the intermediate tissue between the balloons with subsequent fluids delivered via a delivery port within the catheter. The order of fluid washes was three washes of 45 mL saline, followed by one wash of 0.5% Mucomyst in saline, followed by one wash of 25 mM sodium acetate buffer in 7.5% sucrose pH5.5. After ensuring that the intermediate duodenum section was fully drained, the drug product (highly acidic chitosan-nucleic acid polyplex composition) was delivered to the section via the catheter delivery port and incubated for 60 minutes. Following incubation, the distal and proximal balloons were deflated and then the scope and catheter were removed.

Pig Plasma Collection

Pig plasma was collected by the following procedure. Approximately 5 mL of blood was collected from the ear, saphenous or jugular vein with the animal under sedation into a Vacutainer previously spiked with 50 μl of aprotinin (4.7 units/mg protein, 6.6 units/ml), and then immediately placed on ice and delivered to the laboratory for testing. The plasma was collected by spinning the blood samples at 1000×g for 10 minutes and collecting the supernatant. Collected plasma was stored at −80 C until ready for analysis.

Results

Pig plasma SEAP detected in response to administration of c150 chitosan-nucleic acid particles containing gWIZ-SEAP plasmid DNA. Drug product formulation for pH 4 was C(24,98)-N20-c150-Ac25-Suc9-pH4.0. Drug product formulation for pH 4.8 was C(24,98)-N20-c150-Ac25-Suc9-pH4.8.

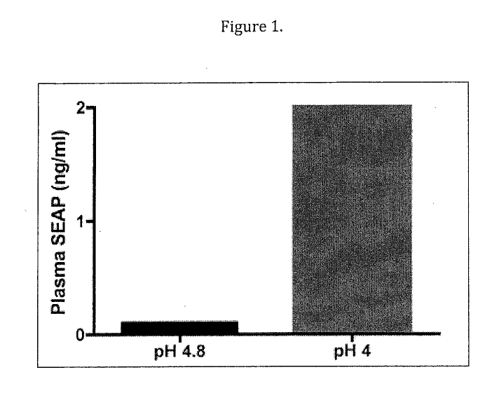

The highly acidic chitosan-nucleic acid polyplex composition with a pH of 4.0 exhibited a substantially higher transfection efficiency in vivo than the pH 4.8 composition as evidenced by the higher level of SEAP in plasma. (FIG. 1).

### Example 10

### Transfection of Mouse Bladder In Vivo

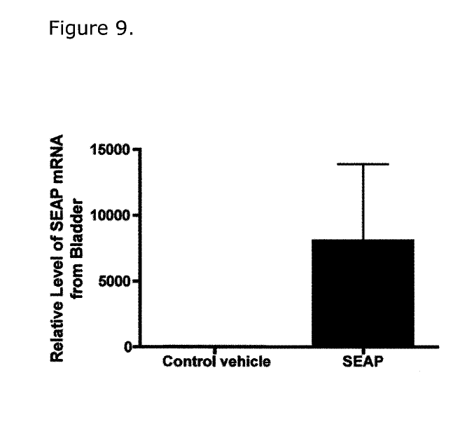

Naïve C57BL/6 mice were delivered with chitosan-DNA polyplexes C(24,98)-c1000-pH4 carrying EF1a-SEAP or control vehicle. After 2 days, mice were sacrificed and tissues were harvested. Relative increases in SEAP mRNA in bladder tissue of the treated mice over naïve mice (non-transfected) are shown in FIG. 9.

Methods

Surgical incisions by laparotomy were made in the abdomen of C57BL/6 female mice to expose and isolate the bladder. Urine was removed followed by delivery with 100 ul of c1000 C(24,98) chitosan polyplex at pH4 carrying the EF1a-SEAP or control plasmid. Two days post-delivery, bladder tissue was collected for RNA extraction followed by RT-qPCR analysis for SEAP mRNA expression.

Results

The highly acidic chitosan-nucleic acid polyplex composition was able to efficiently transfect cells of the bladder in vivo.

### Example 11

### Repeat Dosing Efficacy in Chronic IBD Model

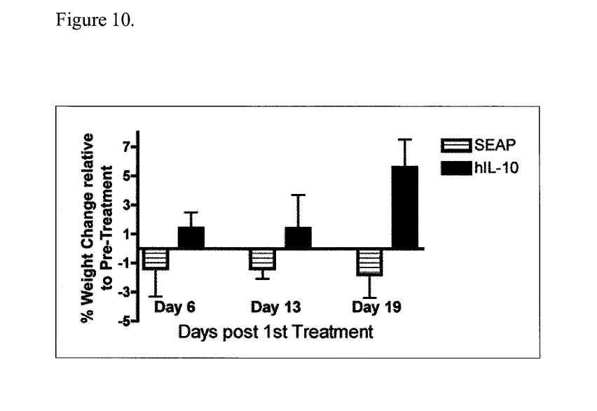

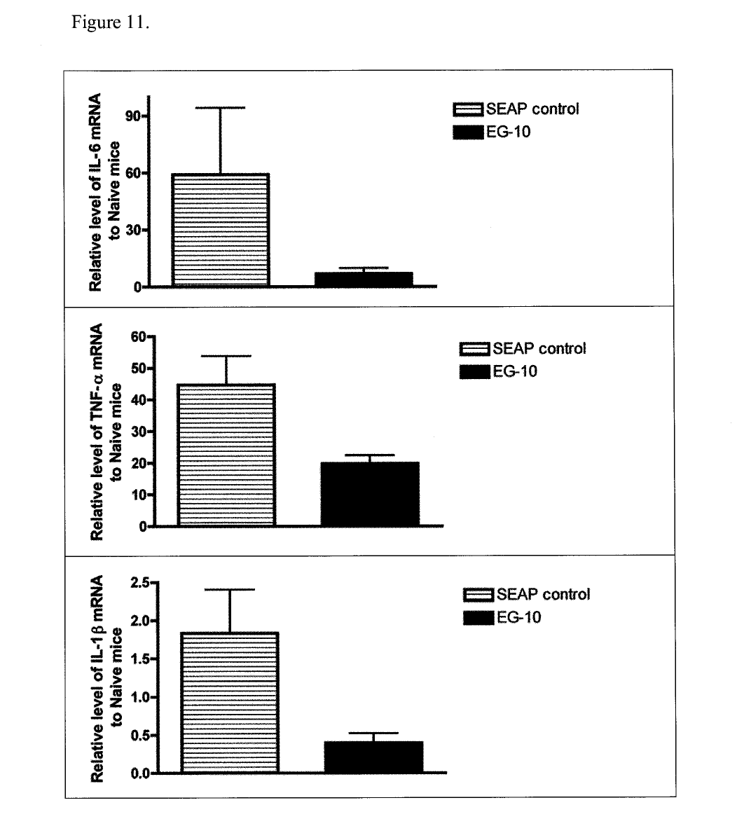

We initiated a repeat dosing study using IL-10 deficient mice that developed chronic colitis naturally. These mice were monitored for symptoms of colitis development weekly. After development of colitis was confirmed (eg. loose and bloody stool), we administered to these mice 3 doses of EG-10 or SEAP (control) nanoparticles via enema. Each dose of nanoparticles was administered 7 days apart. Body weight of these mice were monitored weekly throughout the experiment and significant improvement in weight gain associated with the EG-10 treated group following each weekly treatment were observed (FIG. 10). Five days after the last treatment, mice from both groups were sacrificed and their colons were removed and pro-inflammatory cytokine levels were measured. The EG-10 treated mice resulted in reduced levels of IL-6, IL-1β, and TNF-α mRNA when compared to SEAP treated mice (FIG. 11). These data combined clearly demonstrated the feasibility of multiple dosing and improved therapeutic efficacy of EG-10 in chronic mouse IBD model.

FIG. 10: Effect of EG-10 (hIL-10) on body weight of chronic IBD mice. IL-10-deficient mice with spontaneously developed colitis (at ˜30 weeks of age) were treated with 3 doses of EG-10 or SEAP nanoparticles (control) given by enema 7 days apart. The body weight of each mouse was measured weekly and compared to its own body weight prior to the first treatment (expressed in % weight change). Drug product formulation for both nanoparticles was C(24,98)-N10-c1000-Ac70-Suc9-pH4.0. EG-10 nanoparticles comprised a DNA plasmid with a human interleukin-10 gene (hIL-10) under the control of an elongation factor 1-alpha promoter (EF1a). SEAP (control) nanoparticles comprised a DNA plasmid with a secretable embryonic alkaline phosphatase gene (SEAP) under the control of elongation factor 1-alpha promoter (EF1a).

FIG. 11: Effect of EG-10 (hIL-10) nanoparticles on three pro-inflammatory cytokines. IL-10-deficient mice with spontaneously developed colitis (at ˜30 weeks of age) were treated with 3 doses of EG-10 or SEAP (control) nanoparticles given by enema 7 days apart. Five days after the last treatment, pro-inflammatory cytokine levels were measured in the colons of sacrificed mice: IL-6, TNF-α and IL-β. Drug product formulation for both nanoparticles was C(24,98)-N10-c1000-Ac70-Suc9-pH4.0. EG-10 nanoparticles comprised a DNA plasmid with a human interleukin-10 gene (hIL-10) under the control of an elongation factor 1-alpha promoter (EF1a). SEAP (control) nanoparticles comprised a DNA plasmid with a secretable embryonic alkaline phosphatase gene (SEAP) under the control of elongation factor 1-alpha promoter (EF1a).

### Example 12

### In Vivo Mouse Transfection to Treat COPD or Asthma: Polyplex Delivery to Lung

For establishing mouse COPD models, mice are exposed to cigarette smoke for a duration of 4 to 5 days to establish sub-acute exposure, or for a duration of 6 months to establish chronic exposure, either through a nose-only exposure system or via a smoke chamber, as previously described (see, for example, Fortin et al., 2009, A multi-target antisense approach against PDE4 and PDE7 reduces smoke-induced lung inflammation in mice. Respir Res. 2009 May 20; 10:39.; Miller et al., 2009, Adiponectin and functional adiponectin receptor 1 are expressed by airway epithelial cells in chronic obstructive pulmonary disease. J Immunol. 2009 Jan. 1; 182(1):684-91.; Bonneau et al., 2006, Effect of adenosine A2A receptor activation in murine models of respiratory disorders. Am J Physiol Lung Cell Mol Physiol. 2006 May; 290(5):L1036-43. Epub 2005 Dec. 9.; Brusselle et al., Murine models of COPD. Pulm Pharmacol Ther. 2006; 19(3):155-65. Epub 2005 Aug. 3.; and D'hulst et al., 2005, Time course of cigarette smoke-induced pulmonary inflammation in mice. Eur Respir J. 2005 August; 26(2):204-13.). A mouse COPD model can also be established by exposing trachea to porcine pancreatic elastase for duration of 4 to 5 weeks as described previously (see, for example, Cheng et al., 2009, Prevention of elastase-induced emphysema in placenta growth factor knock-out mice. Respir Res. 2009 Nov. 23; 10:115.; and Pang et al., 2008, Diminished ICAM-1 expression and impaired pulmonary clearance of nontypeable Haemophilus influenzae in a mouse model of chronic obstructive pulmonary disease/emphysema. Infect Immun. 2008 November; 76(11):4959-67. Epub 2008 Sep. 15.).

For establishing mouse asthma models, mice are injected intraperitoneally with chicken ovalbumin mixed with aluminum hydroxide. Days after initial injection, mice are challenged with ovalbumin intranasally as previously described (Bonneau et al. 2006, supra; and Boulares et al., 2003, Gene Knockout or Pharmacological Inhibition of Poly(ADP-Ribose) Polymerase-1 Prevents Lung Inflammation in a Murine Model of Asthma. American Journal of Respiratory Cell and Molecular Biology. Vol. 28, pp. 322-329)

To treat a COPD or asthma model, a highly acidic chitosan-DNA polyplex composition comprising a therapeutic nucleic acid encoding an anti-inflammatory protein is used. Anti-inflammatory proteins are well known in the art. Exemplary anti-inflammatory proteins are reported in the references in Table 14. All references are expressly incorporated herein in their entirety by reference. The highly acidic chitosan-DNA polyplex composition is administered to the lung intranasally or intratracheally under anesthetic (for example, see Dow et al., 1999, infra; and Hogan et al., 1998, infra). At various time points, mice are sacrificed and their lung tissue are collected and processed for transgene mRNA expression and the expression of various cytokines (for example, see Dow et al., 1999, infra; and Hogan et al, 1998, infra). DNA alone is injected alone as control. Intranasal/intratracheal delivery of the highly acidic chitosan-DNA polyplex composition results in significantly increased anti-inflammatory gene mRNA expression in lung cells in vivo and mediates a reduction of the pro-inflammatory cytokine profile.

### Example 13

### In Vivo Mouse Transfection: Polyplex Delivery to Bladder to Treat Cystitis

For establishing Cystitis models, mice or rats may be used. For example, mice are placed under anesthetic and the urethra is cannulated with polyethylene catheter. Following aspiration of urine, the bladder are instilled with acid to induce cystitis as previously described (see, for example, Kirimoto et al. 2007, Beneficial effects of suplatast tosilate (IPD-1151T) in a rat cystitis model induced by intravesical hydrochloric acid. BJU Int. 2007 October; 100(4):935-9. Epub 2007 Aug. 20.; and Chuang et al., 2003, Gene therapy for bladder pain with gene gun particle encoding pro-opiomelanocortin cDNA. J Urol. 2003 November; 170(5):2044-8.).

To treat cystitis, mice are anesthetized and a highly acidic chitosan-DNA polyplex composition comprising a therapeutic nucleic acid encoding an anti-inflammatory protein is administered to the bladder intravesicularly through urethra catheter (see, for example, Kirimoto et al. 2007, supra; and Chuang et al., 2003, supra). Exemplary anti-inflammatory proteins are reported in the references in Table 14. AU references are expressly incorporated herein in their entirety by reference. At various time points, mice are sacrificed and their bladder tissues are collected and processed for histology and transgene mRNA expression. In addition, the expression of various cytokines is examined. DNA alone is injected alone as control. Intravesicular delivery of chitosan-DNA polyplex results in significantly increased anti-inflammatory gene mRNA expression in bladder tissues in vivo and mediates a reduction of the pro-inflammatory cytokine profile.

TABLE 14
Exemplary Anti-Inflammatory Proteins

| GENE | REFERENCE |
|---|---|
| IL-10 | Fedorak et al., 2000, Recombinant human interleukin 10 in the |
| | treatment of patients with mild to moderately active Crohn's |
| | disease. The Interleukin 10 Inflammatory Bowel Disease |
| | Cooperative Study Group, Gastroenterology. 2000 |
| | December; 119(6): 1473-82.; |
| | Whalen et al., 1999, Adenoviral transfer of the viral IL-10 gene |
| | periarticularly to mouse paws suppresses development of |
| | collagen-induced arthritis in both injected and uninjected paws. J |
| | Immunol. 1999 Mar. 15; 162(6): 3625-32. |
| IL-1Ra | Arend et al., 1998, Interleukin-1 receptor antagonist: role in |
| | biology. Annu Rev Immunol. 1998; 16: 27-55.; |
| | Makarov et al., 1996, Suppression of experimental arthritis by |
| | gene transfer of interleukin 1 receptor antagonist cDNA. Proc Natl |
| | Acad Sci USA. 1996 Jan. 9; 93(1): 402-6. |
| IL-1Ra-Ig | Ghivizzani et al., 1998, Direct adenovirus-mediated gene transfer |
| | of interleukin 1 and tumor necrosis factor alpha soluble receptors |
| | to rabbit knees with experimental arthritis has local and distal anti- |
| | arthritic effects. Proc Natl Acad Sci USA. 1998 Apr. |
| | 14; 95(8): 4613-8. |
| IL-4 | Hogaboam et al., 1997, Therapeutic effects of interleukin-4 gene |
| | transfer in experimental inflammatory bowel disease. J Clin Invest. |
| | 1997 Dec. 1; 100(11): 2766-76. |
| IL-17 soluble Receptor | Zhang et al., 2006, Critical role of IL-17 receptor signaling in acute |
| | TNBS-induced colitis. Inflamm Bowel Dis. 2006 May; 12(5): 382-8.; |
| | Ye et al., 2001, Requirement of Interleukin 17 Receptor Signaling |
| | for Lung Cxc Chemokine and Granulocyte Colony-Stimulating |
| | Factor Expression, Neutrophil Recruitment, and Host Defense. |
| | The Journal of Experimental Medicine, Volume 194, Number 4, |
| | Aug. 20, 2001 519-528 |
| IL-6 | Xing et al., 1998, IL-6 is an antiinflammatory cytokine required for |
| | controlling local or systemic acute inflammatory responses. J Clin |
| | Invest. 1998 Jan. 15; 101(2): 311-20. |
| IL-11 | Trepicchio et al., 1997, IL-11 regulates macrophage effector |
| | function through the inhibition of nuclear factor-kappaB. J |
| | Immunol. 1997 Dec. 1; 159(11): 5661-70. |
| IL-13 | Mulligan et al., 1997, Protective effects of IL-4, IL-10, IL-12, and |
| | IL-13 in IgG immune complex-induced lung injury: role of |
| | endogenous IL-12. J Immunol. 1997 Oct. 1; 159(7): 3483-9.; |
| | Muchamuel et al., 1997, IL-13 protects mice from |
| | lipopolysaccharide-induced lethal endotoxemia: correlation with |
| | down-modulation of TNF-alpha, IFN-gamma, and IL-12 production. |
| | J Immunol. 1997 Mar. 15; 158(6): 2898-903. |
| IL-18 soluble Receptor | Aizawa et al., 1999, Cloning and expression of interleukin-18 |
| | binding protein. FEBS Lett. 1999 Feb. 26; 445(2-3): 338-42. |
| TNF-α soluble Receptor | Watts et al., 1999, Soluble TNF-alpha receptors bind and |
| | neutralize over-expressed transmembrane TNF-alpha on |
| | macrophages, but do not inhibit its processing. J Leukoc Biol. |
| | 1999 December; 66(6): 1005-13. |
| TNF-α receptor-IG | Ghivizzani et al., 1998, Direct adenovirus-mediated gene transfer |
| | of interleukin 1 and tumor necrosis factor alpha soluble receptors |
| | to rabbit knees with experimental arthritis has local and distal anti- |
| | arthritic effects. Proc Natl Acad Sci USA. 1998 Apr. |
| | 14; 95(8): 4613-8. |
| TGF-β | Song et al., 1998, Plasmid DNA encoding transforming growth |
| | factor-beta1 suppresses chronic disease in a streptococcal cell |
| | wall-induced arthritis model. J Clin Invest. 1998 Jun. |
| | 15; 101(12): 2615-21.; |
| | Giladi et al., 1994 |
| IL-12 | Hogan et al., 1998, Mucosal IL-12 gene delivery inhibits allergic |
| | airways disease and restores local antiviral immunity. Eur J |
| | Immunol. 1998 February; 28(2): 413-23. |
| IFN-γ | Dow et al., 1999, Systemic and local interferon gamma gene |
| | delivery to the lungs for treatment of allergen-induced airway |
| | hyperresponsiveness in mice. Hum Gene Ther. 1999 Aug. |
| | 10; 10(12): 1905-14. |
| IL-4 soluble Receptor | Steinke et al., 2001, Th2 cytokines and asthma. Interleukin-4: its |
| | role in the pathogenesis of asthma, and targeting it for asthma |
| | treatment with interleukin-4 receptor antagonists. Respir Res. |
| | 2001; 2(2): 66-70. Epub 2001 Feb. 19. |

All citations are expressly incorporated herein in their entirety by reference.

We claim:

1. A highly acidic chitosan-nucleic acid polyplex composition, comprising stable chitosan-nucleic acid polyplexes, wherein said composition has a pH below 4.5.

2. The composition according to claim 1, wherein said composition has a pH below 4.2.

3. The composition according to claim 1, wherein said composition has a pH below 4.0.

4. The composition according to claim 1, wherein said composition has a pH below 3.8.

5. The composition according to claim 1, comprising a counter anion concentration of between 10-200 mM.

6. The composition according to claim 5, wherein the counter anion is acetate.

7. The composition according to claim 1, having a nucleic acid concentration of at least 0.5 mg/ml.

8. The composition according to claim 1, having a nucleic acid concentration of at least 1.0 mg/ml.

9. The composition according to claim 1, having a nucleic acid concentration of at least 1.5 mg/ml.

10. The composition according to claim 1, where said composition is free of polyplex precipitate.

11. The composition according to claim 1, wherein said chitosan-nucleic acid polyplexes comprise a therapeutic nucleic acid construct.

12. A method of transfecting cells of a mucosal epithelium, comprising contacting said cells of a mucosal epithelium with the composition according to claim 1.

13. The method according to claim 12, wherein said mucosal epithelium is present in a tissue selected from the group consisting of gastrointestinal tract tissue, respiratory tract tissue, lung tissue, sinus cavity tissue, oral cavity tissue, urinary tract tissue, bladder tissue, vaginal tissue, uterine tissue, cervical tissue, eye tissue, esophagus tissue, salivary gland tissue, nasolaryngeal tissue, kidney tissue, and larynx/pharynx tissue.

14. The method according to claim 12, wherein said mucosal epithelium is present in gastrointestinal tract tissue.

15. The method according to claim 12, wherein said mucosal epithelium is present in bladder tissue.

16. The method according to claim 12, wherein said mucosal epithelium is present in lung tissue.

17. A pharmaceutical composition, comprising the composition according to claim 13, wherein said pharmaceutical composition has a pH less than 4.5.

18. The pharmaceutical composition according to claim 14, wherein said pharmaceutical composition is isotonic.

19. A method for treating a disease involving inflammation of a mucosal epithelium, comprising administering to a patient having a disease involving inflammation of a mucosal epithelium a therapeutically effective amount of the pharmaceutical composition according to claim 17, wherein said therapeutic nucleic acid construct encodes an anti-inflammatory protein, and wherein said pharmaceutical composition is administered locally to said mucosal epithelium.

20. The method according to claim 19, wherein said anti-inflammatory protein is selected from the group consisting of TNFα inhibitors, IL-1 inhibitors, and IL-10.

21. The method according to claim 19, wherein said anti-inflammatory protein is IL-10.

22. The method according to claim 19, wherein said disease is IBD.

23. The method according to claim 19, wherein said disease is interstitial cystitis.

24. The method according to claim 19, wherein said disease is COPD.

25. The method according to claim 19, wherein said disease is asthma.
